(19) United States
(12) Patent Application Publication
(10) Pub. No.: US 2024/0279152 A1
(43) Pub. Date: Aug. 22, 2024

(54) METHODS OF PRODUCING DIALKYL MALONATE ESTERS AND USES THEREOF

(71) Applicant: CARGILL, INCORPORATED, Wayzata, MN (US)

(72) Inventors: Timothy Walter ABRAHAM, Minnetonka, MN (US); Ravi R. GOKARN, Omaha, NE (US); Nolan Ray Mente, Pella, IA (US); Ryan Edward Patet, Bloomington, MN (US)

(73) Assignee: CARGILL, INCORPORATED, Wayzata, MN (US)

(21) Appl. No.: 18/564,916

(22) PCT Filed: May 19, 2022
(86) PCT No.: PCT/US2022/072426

§ 371 Date: Nov. 28, 2023

Related U.S. Application Data
(60) Provisional application No. 63/194,582, filed on May 28, 2021.

(51) Int. Cl. C07C 67/08; C07C 67/54

(52) U.S. Cl. CPC C07C 67/08; C07C 67/54

(57) ABSTRACT

The present disclosure relates to a method for making a dialkyl malonate ester, including a) acidifying a malonate composition comprising:
at least 30 wt % based on malonic acid equivalents of a compound of formula I:

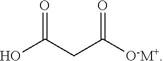

where M⁺ is a Group I alkali metal cation or ammonium cation. The malonate composition optionally includes:
   a compound of formula II:

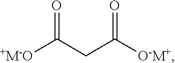

and/or
malonic acid. Step a) comprises contacting an organic alcohol and an inorganic acid with the malonate composition to form a first acidified malonate composition comprising:
   a compound of formula IV:

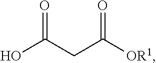

a compound of formula V:

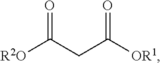

and/or
malonic acid. R¹ and R² are independently (C₁-C₈)alkyl.

### CROSS-REFERENCE TO RELATED APPLICATIONS

This application claims the benefit of U.S. Provisional Patent Application No. 63/194,582, filed May 28, 2021, which is incorporated by reference herein in its entirety.

### FIELD OF THE DISCLOSURE

The disclosure relates to a process for producing dialkyl malonate esters. The dialkyl malonate esters can be formed from malonates that are recovered from a fermentation process.

### BACKGROUND

Fermentation processes are used commercially at large scales to produce organic molecules such as ethanol, citric acid and lactic acid. In those processes, a carbohydrate is fed to an organism that metabolizes the carbohydrate to a desired fermentation product. The carbohydrate and organism are selected together so that the organism can efficiently use the carbohydrate to form the desired product in good yield. It is becoming more common to use genetically engineered organisms in these processes, in order to optimize yields and process variables, or to enable a particular fermentation product to be produced.

Fermentation processes have also been applied to produce reactants that can be used to produce a product of interest. However, depending on the specifics of the fermentation method, it can be difficult to produce and isolate one specific reactant. Thus, before a fermentation product can be used as a reactant additional purification steps may need to be taken. This can add time and costs. It is, therefore, desirable to find methods to quickly and efficiently produce fermentation products as reactants to efficiently produce a desired product.

### SUMMARY OF THE DISCLOSURE

Various embodiments of the present disclosure relate to a method for making a dialkyl malonate ester. The method includes a) acidifying a malonate composition comprising:
at least 30 wt % based on malonic acid equivalents of a compound of formula I:

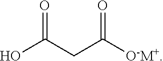

In formula I, M⁺ is a Group I alkali metal cation or ammonium cation. The malonate composition optionally further includes at least one of:
a compound of formula II:

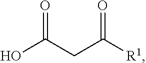

and
malonic acid. The acidifying of the malonate composition comprises contacting an organic alcohol and an inorganic acid with the malonate composition to solubilize the malonate composition to form a first acidified malonate composition comprising at least one of:
a compound of formula IV

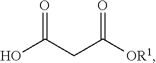

a compound of formula V

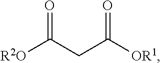

and
malonic acid. R¹ and R² are independently (C₁-C₈)alkyl. The (C₁-C₈) alkyl group can be linear, branched or cyclic (e.g., a cycloalkyl). The method further includes optionally heating the malonate composition, the first acidified malonate composition, or both, to form the dialkyl malonate ester. The method further includes step b) which includes separating a precipitate comprising M⁺ from the product of step a) to form a second acidified malonate composition comprising the compound of formula IV the compound of formula V, malonic acid, or a mixture thereof, and optionally heating the second acidified malonate composition to form the dialkyl malonate ester, wherein the optional heating of step a), step b), or both, is performed to form the dialkyl malonate ester. The method further includes step c) which includes distilling the product of step b) to isolate a distillate comprising the dialkyl malonate ester, wherein the dialkyl malonate ester is greater than or equal to 90 wt % of the distillate (for example, 90 wt % to 100 wt % or 95 wt % to 99.9 wt % or 95 wt % to 99.5 wt %).

According to various embodiments, the malonate composition of step a) can be a reaction product of a fermentation method.

According to various embodiments the organic alcohol(s) used can be methanol or ethanol.

According to various embodiments, the inorganic acid used can be sulfuric acid.

According to various embodiments, the dialkyl malonate ester can be a dimethyl malonate ester or diethyl malonate ester.

### BRIEF DESCRIPTION OF THE FIGURES

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is block diagram showing a process for making a dialkyl malonate ester.

FIG. 2 is a schematic diagram of a batch reactor.

FIG. 3 is a graph showing kinetic parameters of the conversion of malonic acid to esters of malonic acid of Example 6.

FIG. 4 is schematic diagram of a batch reactor of Example 6.

FIGS. 5A and 5B are graphs showing the profile of the reaction system of Example 6 until a 99 mol % yield of diethyl malonate ester is produced.

FIG. 6 is a graph showing the profile of another reaction system of Example 6 until a 99 mol % yield of diethyl malonate ester is produced.

FIG. 7 is a schematic diagram showing a distillation column arrangement according to Example 7.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In this document, the term “alkyl” is used to mean acyclic or cyclic groups having from 1 to 20 carbon atoms ((C₁-C₂₀)), 10 to 20 carbon atoms ((C₁₀-C₂₀)), 12 to 18 carbon atoms ((C₁₂-C₁₈)), 6 to about 10 carbon atoms ((C₆-C₁₀)), 1 to 10 carbons atoms ((C₁-C₁₀)), 1 to 8 carbon atoms ((C₁-C₈)), 2 to 8 carbon atoms ((C₂-C₈)), 3 to 8 carbon atoms ((C₃-C₈)), 4 to 8 carbon atoms ((C₄-C₈)), 5 to 8 carbon atoms ((C₅-C₈)), 1 to 6 carbon atoms ((C₁-C₆)), 2 to 6 carbon atoms ((C₂-C₆)), 3 to 6 carbon atoms ((C₃-C₆)), 1 to 3 carbon atoms ((C₁-C₃)). or 1 to 2 carbon atoms ((C₁-C₂)). Examples of (C₁-C₂₀)-alkyl groups include acyclic groups such as those with from 1 to 8 carbon atoms such as methyl (i.e., CH₃), ethyl, n-propyl, n-butyl, n-pentyl, n-hexyl, n-heptyl, n-octyl groups. Examples of (C₁-C₂₀)-alkyl groups include cyclic groups such as those with from 1 to 6 carbon atoms such as cyclopropyl, cyclobutyl, cyclopentyl, and cyclohexyl groups.

Described herein is a process for making a dialkyl malonate ester. Examples of preferred dialkyl malonate esters include a (C₁-C₈)dialkyl malonate ester, (C₁-C₆)dialkyl malonate ester, a (C₁-C₄)dialkyl malonate ester), and a (C₁-C₃)dialkyl malonate ester). Examples of particularly preferred dialkyl malonate esters that can be made using the disclosed methods can include a diethyl malonate ester and dimethyl malonate ester. Diethyl malonate ester can be an especially preferred dialkyl malonate ester. Products formed according to the methods described herein, for example, diethyl malonate ester, can be ultra-pure. For example, a diethyl malonate ester produced from the disclosed method can have a purity of at least 90%, 95%, 98%, 99%, 99.5%, or greater. Malonic acid and its dialkyl malonate ester derivatives (DEM) are used in the production of many industrial and consumer products, including polyesters, paints and coatings, solvents, electronic products, flavors, fragrances, vitamins, pharmaceuticals, agrochemicals, surgical adhesives, and food additives. As a particular example, the high purity of diethyl malonate ester obtained can be particularly well suited for polymerization reactions to ultimately be used as a material in an electronic component.

The diethyl malonate ester formed according to the instantly described process uses a malonate composition as a starting material. As used herein, the term “malonate” or “malonate composition” includes malonic acid (formula (III), below) and salts thereof. Malonic acid equivalents, as used herein, means the sum of: monosalts of malonic acid (formula I), disalts of malonic acid (formula II), and free malonic acid (formula III) excluding metals ions and other cations. When referring to malonate concentration (e.g. malonate recovered) herein, we are referring to the concentration of malonic acid equivalents.

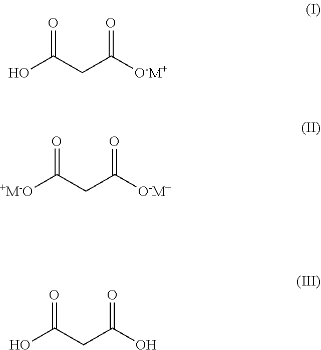

In formulas I and II, M is a Group I alkali metal cation (such as Li⁺, Na⁺, K⁺, ammonium (such as NH₄⁺), or mixtures, thereof.

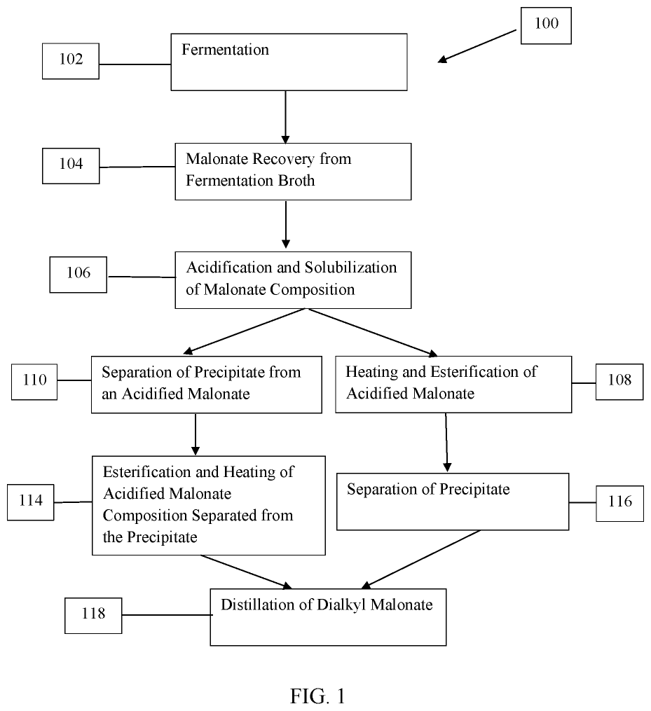

The overall process 100 for making the dialkyl malonate ester is shown in block diagram form in FIG. 1. The malonate composition can be a product of a fermentation process (step 102) and is subsequently recovered from a fermentation broth (step 104). As used herein, the term “fermentation broth” generally means a mixture derived from a microbial fermentation process, which contains a medium (liquid; comprising, for example, the produced malonate composition and other organic acids, salts, metals, residual sugars, and other fermentation byproducts) and biomass (solid; comprising, for example, cells and cell debris).

As used herein, the term “fermentation” or “fermenting” generally means the feeding of a carbon source (e.g., a sugar, such as glucose) to a microorganism under conditions that enable the microorganism to use the carbon source to produce the malonate composition.

Microorganisms that can be used to produce the malonate composition include any suitable microorganism capable of producing malonic acid from fermentable carbon sources (e.g., glucose, sucrose, and/or other carbohydrates). Representative examples of microorganisms include, but are not limited to, yeasts, such as Saccharomyces cerevisiae, Kluyveromyces lactis, Kluyveromyces marxianus, Yarrowia lipolytica, Pichia kudriavzevii (alternatively referred to as Candida krusei and Issatchenkia orientalis), Schizosaccharomyces pombe, or combinations thereof that are adapted for the production of the malonate composition, for example, genetically engineered versions thereof. In some examples, the host cell can include a microorganism such as a bacterium. Examples of suitable bacteria include Streptococcus, Lactobacillus, Bacillus, Escherichia, Salmonella, Neisseria, Acetobactor, Arthrobacter, Aspergillus, Bifidobacterium, Corynebacterium, Pseudomonas, or a mixture thereof. A genetically modified Pichia kudriavzevii that has been adapted to make significant amounts malonate composition is particularly preferred for low pH fermentation performance and high malonate composition tolerance. Preferably, acid tolerant organisms, such as, Kluyveromyces marxianus, Yarrowia lipolytica, Pichia kudriavzevii (alternatively referred to as Candida krusei and Issatchenkia orientalis), Schizosaccharomyces pombe, and filamentous fungi, such as fungi of the genus Aspergillus are utilized. Saccharomyces cerevisiae and Kluyveromyces lactis are preferred in some instances due to the relative ease of conducting genetic modifications to them and the ready availability of genetic tools for modifying them.

The fermentation that provides the malonate composition recovered using the methods described herein can be conducted under any suitable conditions. For example, a fermenter, can be used to produce a malonate-containing fermentation broth using an organism adapted to make the malonate composition. Such fermentation vessels and associated equipment, such as seed vessels are known in the art. The general fermentation process can utilize any number of commercially available carbohydrate substrates, such carbohydrate substrates comprising high levels of glucose and/or sucrose (for example corn syrups containing at least 40 wt %, at least 50 wt %, at least 60 wt %, at least 70 wt %, at least 80 wt %, at least 85 wt %, at least 90 wt %, or at least 95 wt % glucose by dry weight, and typically less than 99 wt % glucose, for example less than 98 wt %). Fermentation methodology is well-known in the art. For example, the fermentation can be carried out in a batch-wise, fed-batch, continuous or semi-continuous manner.

The fermentation broth during the fermentation typically includes a fermentable carbon source (e.g. the carbohydrate substrates described above), and, typically, a source of nitrogen (such as amino acids, proteins, inorganic nitrogen sources such as urea, ammonia or ammonium salts, and the like), phosphates and sulfates, and additional media components such as vitamins, salts (for example metals), and other materials that can improve cellular growth and/or malonate fermentation production, and water. These components can be fed into a fermenter to conduct the fermentation (e.g., promote growth and production of the malonate composition). The microbial culture can be grown under aerobic or micro-aerobic conditions provided by sparging an oxygen containing gas (e.g., air or the like). At the end of the fermentation, all but small quantities of the carbon source have been used by the microorganism, and most of the vitamins and, salts and other materials have been used with the main resulting material at the end of fermentation being the malonate composition, ions (such as metal ions, ammonium ions, sulfates and phosphate), water and cell biomass, organic byproducts (such as organic acids, hydroxyorganic acids and polyols) and other residual materials as further described below.

Typically, at the start or soon after the start of the fermentation, 3 to 15 g/L of ammonium sulfate is present; about 1-6 g/L phosphate anion (e.g. added as potassium dihydrogen phosphate) is present, 0.25 to 4 g/L magnesium sulfate, trace elements, vitamins and 75 g/L to 200 g/L glucose, for example, 130 g/L to 180 g/L glucose. In some instances, small quantities of calcium cations may be added during the fermentation. Typically, the amount of calcium cations present in the fermentation broth at the end of the fermentation is less than 1 wt %, preferably less than 0.5 wt %, (for example, less than 0.1 wt %), and more preferably less than 0.05 wt % calcium cations. As discussed in greater detail herein, the pH can be allowed to range freely during the production of the malonate composition, or may be buffered, if necessary, to prevent the pH from falling below or rising above predetermined levels. Typically, the pH of the fermentation broth at the end of the fermentation can be less than 7.0, less than 6.0, less than 5.0, less than 4.0, less than 3.0, typically from 2.0 to 7.0, from 2.5 to 5.0, from 2.7 to 5.0, from 3.0 to 5.0, from 3.5 to 4.5, from 3.0 to 4.0, or from 3.0 to 3.5. Preferably the pH of the fermentation broth at the end/completion of the fermentation is within 1 pH unit below and 2 pH units above the pH used during the recovery of the malonate composition, and more preferably within 1 pH unit of the pH used during the recovery of the malonate composition (for example, if the recovery of the first crop of the malonate composition takes place at a pH of 4.0, then the fermentation pH at the end of fermentation preferably is from 3.0 to 6.0, and more preferably a pH of from 3 to 5). The fermentation can be carried out to allow the pH of the fermentation to drop into a preferred range as the malonate composition is produced. The pH of the fermentation broth can thereafter be maintained within the desired pH range. The desired pH range may vary depending on the extent of the fermentation and the final pH target. For example, the pH may be maintained at a relatively high pH through the addition of basic materials and then allowed to decline as additional malonate composition is produced until toward the end of the fermentation the desired final pH for the fermentation broth is reached.

The fermentation typically is conducted aerobically or microaerobically, depending on the organism utilized and the requirements of the pathway used to make the malonate composition. If desired, oxygen uptake rate (OUR) can be varied throughout fermentation as a process control. At least partially due to their responsiveness to varying the OUR, crabtree negative organisms, such as crabtree negative yeasts are preferred for use in making the malonate composition. Kluyveromyces marxianus, Yarrowia lipolytica, Pichia kudriavzevii (alternatively referred to as Candida krusei and Issatchenkia orientalis), and Schizosaccharomyces pombe, are examples of crabtree negative yeasts that are preferable to use for the manufacture of the malonate composition.

In one example, the concentration of cells of the organism used to make the malonate composition typically are present in the fermentation broth at the end of fermentation typically in the range of from 0.5 to 30, from 2 to 30, or from 3 to 20 g dry cells/L of fermentation broth. Typically, these cells are removed prior to recovering the malonate composition. Preferably, the cells are removed after sufficient water has been removed to create an aqueous solution of typically from 10 to 30 percent by weight the malonate composition, for example from 15 to 30 percent by weight malonate composition, as further described below. Preferably, after the biomass containing the cells have been removed, additional water is removed to create an aqueous concentrate having at least 400 g malonate composition/kilogram solution. Alternatively, the first aqueous concentrate can be created by removing the water in a single or multiple steps carried out after the biomass/cells are removed, as described below.

The final yield of the malonic acid equivalents of the malonate composition produced during the fermentation based on the starting carbon source typically is at least 25%, at least 30%, at least 40%, at least 50%, or greater than 70%. The titer at the completion of the fermentation typically is at least 30 g/L, at least 40 g/L, at least 50 g/L, at least 70 g/L, at least 80 g/L, at least 100 g/L, at least 110 g/L, at least 120 g/L, at least 130 g/L, at least 140 g/L, at least 150 g/L, at least 160 g/L, for example, in a range of from 30 g/L to 300 g/L, from 40 g/L to 300 g/L, from 60 g/L to 250 g/L, from 80 g/L to 220 g/L, or from 100 g/L to 150 g/L, at the end of the fermentation.

The Group I alkali metal cation and/or ammonium cation used in the recovery of the malonate composition can be added after the fermentation, or at least some or all of the required Group I alkali metal cation and/or ammonium cation can be added during the fermentation. For example, the fermentation broth can include a source of the Group I alkali metal cation, such as a Group I alkali metal carbonate, Group I alkali metal bicarbonate, Group I alkali metal oxide or a Group I alkali metal hydroxide for pH control during the fermentation. The Group I alkali metal carbonate, Group I alkali metal bicarbonate, Group I alkali metal oxide or a Group I alkali metal hydroxide can help maintain the pH of the fermentation broth at the levels described above. A source of ammonium cations such as ammonia, ammonium hydroxide, ammonium bicarbonate, ammonium carbonate and the like can be added as a source of nitrogen for the fermentation and to adjust or maintain the pH during the fermentation.

As used herein, the term “Group I alkali metal cations” generally means, for example, cations of lithium (Li), sodium (Na), and potassium (K); that is to Li⁺, Na⁺, and K⁺. Further, as used herein, the term “ammonium cation” includes any cation of the formula R₄N⁺, wherein R is hydrogen or alkyl.

Suitable sources of the Group I alkali metal cations include, but are not limited to, sodium hydroxide, potassium hydroxide, potassium carbonate, sodium carbonate, and the like. Suitable sources of ammonium cation include, for example, ammonium hydroxide, ammonium carbonate, and the like.

The source of the Group I alkali metal cation can also be from compounds that do not necessarily control the pH of the fermentation broth or the aqueous solution used for recovering the malonate composition described herein. For example, the Group I alkali metal cation source can be from compounds such as, sodium chloride, potassium chloride, ammonium chloride, and the like. Regardless of the purpose (e.g., pH control) or properties, the Group I alkali metal cation source can be added to the fermentation broth during or at the end of the fermentation, or to the aqueous solution used for recovering the malonate composition described herein at any time prior to or during the recovery of the malonate composition.

Removing water from the aqueous solutions (e.g., the fermentation broth and/or the aqueous filtrate) can be performed using any suitable method at any suitable temperature and pressure. In addition, the removing water from the fermentation broth to increase the concentration of the malonate composition and produce an aqueous concentrate can be implemented two or more times as described in greater detail herein. For example, the water can be removed at less than atmospheric pressure, such as at less than 101.325 kPa. In addition, or alternatively, the water typically can be removed at a temperature (e.g., an aqueous bulk temperature) of 100° C. or less, 90° C. or less, 80° C. or less, preferably 70° C. or less, more preferably 60° C. or less, 50° C. or less; from 40° C. to 100° C., from 50° C. to 90° C., from 50° C. to 80° C., from 50° C. to 70° C. In order to minimize the premature crystallization/precipitation of the malonate composition, the temperature of the solution containing the malonate composition is typically maintained at or above 30° C., at least 35° C., at least 40° C., at least 50° C., at least 60° C.; from 30° C. to 100° C., 30° C. to 90° C. or 40° C. to 80° C. if the concentration of the malonate composition is at least 400 g/kg; and preferably, if 500 g/kg or greater malonate composition concentration, at least 40° C., at least 45° C. The temperature of the solution containing the malonate composition preferably is kept lower than 90° C., preferably less than 80° C., and less than 70° C. to minimize decarboxylation of the malonate composition.

Although a lower limit of about 400 g/kg malonate composition is described in connection with the removing of water from the aqueous solution obtained from the fermentation broth comprising the malonate composition to increase the concentration of the malonate composition, the methods described herein also include removing water from the fermentation broth and the aqueous filtrate comprising the malonate composition to enhance the recovery of the malonate composition include increasing the concentration of the malonate composition to at least 400 g/kg; at least 425 g/kg, at least 450 g/kg; at least 475 g/kg, at least 500 g/kg, at least 525 g/kg, at least 550 g/kg, at least 575 g/kg, at least 600 g/kg; less than 800 g/kg, less than 775 g/kg, such as from 400 g/kg to 775 g/kg, from 450 g/kg to 700 g/kg, and from 500 g/kg to 600 g/kg.

The pH of the aqueous concentrate and/or the aqueous filtrate during the recovery of the malonate composition is typically from 2.5 to 5.0, more preferably from 2.5 to 4.5, from 3.0 to 4.0 (for example from 3.0 to 3.5).

Typically, concentration of the malonate composition after a cell removal step is between 30 g/kg and 775 g/kg the malonate composition. Preferably, the cells are removed as and the concentration of the malonate composition after removal step typically is from 30 g/kg to 400 g/kg, 50 g/kg to 350 g/kg, 80 g/kg to 350 g/kg. Or the cells are removed and the concentration of the malonate composition after removal step typically is 400 g/kg to 800 g/kg, 400 g/kg to 700 g/kg, 500 g/kg to 600 g/kg. For example, the biomass can be removed using one or more filtration steps or one or more centrifugation steps. Centrifugation can be carried out in a decanter centrifuge, such as a horizontal-type decanter centrifuge, a disc stack centrifuge, or hydrocyclones.

As mentioned herein, removing water from the aqueous solution to increase the concentration of the malonate composition and produce an aqueous concentrate can be implemented two or more times. For example, removing water can comprise removing water from the fermentation broth and can further comprise removing water to provide a first aqueous concentrate.

The malonate composition can be recovered from a first aqueous concentrate and the second aqueous concentrate by any suitable method including precipitating or crystallizing. The step of recovering the malonate composition typically includes lowering the temperature of the first aqueous concentrate and/or the second aqueous concentrate to below 40° C., below 35° C., below 30° C., below 25° C., below 20° C., below 15° C., below 10° C., below 5° C., below 0° C.; typically from 0° C. to 30° C., from 4° C. to 25° C. Those of skill in the art will recognize that the temperature at which the malonate composition can effectively be recovered, will depend on the concentration of the malonate composition in first aqueous concentrate and/or second aqueous concentrate, the pH of the relevant first aqueous concentrate and/or second aqueous concentrate, the number of recovery steps that are utilized, and the desired overall yield of the malonate composition resulting from the recovery steps. For example, the malonate composition can be recovered at a higher temperature if the relevant aqueous concentrate comprises 700 g/kg of the compound of the malonate composition relative to when the concentration of the malonate composition is 400 g/kg in the aqueous concentrate. However, where a higher yield of the malonate composition is desirable, a lower temperature (typically at or below 30° C. is utilized).

The malonate composition recovered is typically a mixture of malonic acid, a compound of formula I, and potentially a compound of formula II. For example, for pH's during the recovery of the first crop at a pH between 2.5 and 5.0 (preferably from 2.5 and 4.0, for example from 3.0 to 4.0, from 3.0 to 3.5), and with a second crop recovered without adjusting the pH (i.e., the malonate composition in the filtrate is concentrated, but no base or acid is added to adjust the pH). Malonate composition recovered from the first aqueous concentrate typically comprises: at least 50 wt % (for example, at least 55 wt %, at least 60 wt % of the compound of formula 1 (for example from 55 wt % to 75 wt % (from 60 wt % to 75 wt %) of the compound of formula 1; less than 50 wt % (for example, less than 45 wt %, less than 40 wt % (from 1 wt % to 45 wt %, from 2 wt % to 40 wt %, from 20 wt % to 40 wt %)) of malonic acid; and less than 15 wt % (for example, less than 10 wt %, less than 5 wt %, less than 2 wt %, less than 1 wt % (for example from 0.1 wt % to 10 wt %) of the compound of formula II. Malonate composition recovered from the second aqueous concentrate typically comprises: at least 40 wt % (for example, at least 45 wt %, at least 60 wt % (for example from 40 wt % to 90 wt %, from 60 wt % to 90 wt %) of the compound of formula 1; less than 55 wt % (for example, less than 50 wt %, less than 15 wt % for example, from 1 wt % to 55 wt %, from 1 wt % to 15 wt %) of malonic acid; and less than 25 wt % (for example, less than 20 wt %, less than 17 wt % (for example from 0.05 wt % to 20 wt %, from 1 wt % to 17 wt %)) of the compound of formula II. Typically the total the malonate composition recovered (for example from the sum of the first and the second crop comprises: at least 40 percent (for example at least 50 percent, at least 60 percent) by weight of the compound of formula I; no greater than 25 percent (for example no greater than 20 percent, no greater than 15 percent, no greater than 10 percent, or not greater than 5 percent) by weight of the compound of formula II; and less than 50 percent (less than 45 percent, less than 40 percent, less than 35 percent, less than 30 percent) by weight malonic acid.

The pH of the first aqueous concentrate during the recovery of the first crop typically is as described above. Typically, the pH of the second aqueous concentrate during the recovery of the second crop is not adjusted by the addition of an acid or a base. The pH of the second aqueous concentrate during the recovery of the second crop is typically 0.2 to 1.0 pH unit higher than the pH utilized in the recovery of the first crop, more typically a pH during the second crop that is about 0.2 to about 0.6 higher than the pH utilized during the recovery of the first crop.

Lowering the temperature of the first aqueous concentrate (and the second aqueous concentrate if a second crop is recovered) causes the precipitation of the malonate composition from the first aqueous concentrate (and the second aqueous concentrate if a second crop is recovered). The malonate composition can then be removed by, e.g., filtration, to give an aqueous filtrate comprising the malonate composition and a first crop of precipitate of recovered the malonate composition. The aqueous filtrate comprising the malonate composition can, in turn, be concentrated by removing water, as described herein, from the aqueous filtrate to increase the malonate composition concentration typically to a concentration of from 380 g/kg to 775 g/kg (preferably from 400 g/kg to 700 g/kg, for example from 500 g/kg to 600 g/kg malonate composition concentration) to form a second aqueous concentrate.

Removing water to form the second aqueous concentrate can be performed using any suitable method at any suitable temperature and pressure. Preferably, the temperatures and pressured described for use in removing water to form the first aqueous concentrate are utilized.

A second crop of precipitate of the malonate composition typically is recovered from the second aqueous concentrate by lowering the temperature of the second aqueous concentrate to similar temperatures as described above for use in recovering the first crop of the malonate composition. The sum of the amounts of the malonate composition recovered in the first crop plus the second crop based on malonic acid equivalents typically constitute at least 70%, at least 75%, at least 80%, at least 85%, at least 90%, at least 95%; 85% to 99%, 85% to 98% or 90% to 99% of the malonate composition present in the fermentation broth. If no solvent assisted precipitation is used during recovery of the first crop or second crop, the recovery of the malonate composition based on malonic acid equivalents typically is from 70% to 90% of the malonate composition present in the fermentation broth. The higher recoveries (e.g. greater than 85%, at least 90%, for example from 90% to 98%, from 93% to 98% of the malonate composition based on malonic acid equivalents in the fermentation broth recovered) typically are achieved using solvent assisted precipitation during the first crop and/or second crop recovery.

In theory, a third, fourth or even a fifth crop (or more) of precipitate of the malonate composition can be obtained by continuing to remove water from additional aqueous filtrates that contain the malonate composition by concentration of the malonate composition, and lowering the temperatures as set forth for the first crop and second crop of the malonate composition precipitate. As mentioned earlier, the precipitation from during any crop may include crystallization or precipitation by other means known to one of ordinary skill in the art.

As mentioned above, the recovery of the compound of the formula I from any of the first aqueous concentrate and/or the second aqueous concentrate (if present) can, in addition to or instead of lowering the temperature, be accomplished by adding an organic solvent. For example, the methods described herein can include adding an organic solvent to the second aqueous concentrate to enhance the recovery of the malonate composition. Preferably, the organic solvent is water-miscible. As mentioned above, preferably the temperature of the aqueous concentrate is reduced to less than 40° C. to enhance the recovery of the malonate composition. Preferably, a water-miscible organic solvent is added to the second aqueous concentrate to enhance the recovery of the malonate composition from the second aqueous concentrate. In a particularly preferred aspect, a water-miscible organic solvent is added to the second aqueous concentrate, but is not added to the first aqueous concentrate. Suitable water-miscible organic solvents include, but are not limited to alcohols (e.g., ethanol), ketones (e.g., acetone), nitriles (e.g., acetonitrile), ethers (e.g., tetrahydrofuran), and combinations thereof. Suitable alcohols include C₁-C₅ alcohols (preferably C₁-C₃), such as methanol, ethanol, propanol, isopropanol, and the like, and combinations thereof. In some examples, ethanol is the water-miscible organic solvent. In some instances, the water-miscible organic solvent is added prior to recovering the malonate composition from the first and/or second aqueous concentrate.

Alkyl malonate esters can be produced from the malonate composition described herein. As used herein, alkyl malonate esters are understood to mean an esterified reaction product of any of the components of the malonate composition. Forming the alkyl malonate esters begins by acidifying and solubilizing the malonate composition (step 106) to form an acidified malonate composition. Acidifying and solubilizing the malonate composition (in which formula I is typically present at least 30 wt % of the malonate composition) includes contacting the malonate composition with an inorganic acid and an organic alcohol.

Examples of suitable inorganic acids include sulfuric acid, hydrochloric acid, nitric acid, phosphoric acid, and mixtures thereof. Sulfuric acid is typically preferred as the inorganic acid. Sulfuric acid may be preferred due to its ready availability and low cost as well as its ability to present a lower corrosivity risk to reaction hardware than other inorganic acids; Thus, in addition to serving as a catalyst for esterification, the inorganic acid also helps to solubilize the malonate composition in the alcohol by converting the malonate salt into malonic acid. Increased solubilization of the malonate composition can help to increase the ultimate yield of the final product formed from the instantly described process. A pH of the malonate composition during solubilization and acidification is typically brought to a pH of less than or equal to 2 (for example, a pH of 0.1 to 2, a pH of 0.1 to 1.5, or a pH of 0.1 to 1). Bringing the pH to this range results in the composition of formula III being the predominate species present. An amount of inorganic acid added is measured by a molar ratio of equivalents of inorganic acid to equivalents of M⁺. That ratio is from 0.2:1.0 to 2.0:1.0 equivalents of inorganic acid to equivalents of M⁺ or from 0.5:1.0 to 1.5:1.0, 0.7:1.0 to 1.2:1.0.

The organic alcohol added is typically methanol, ethanol, propanol, butanol, pentanol, hexanol, heptanol, or octanol. As understood, alcohols such as propanol, butanol, pentanol, hexanol, heptanol, or octanol can be branched, unbranched, acyclic, or cyclic alcohols. In some preferred aspects, the organic alcohol added is ethanol or methanol. In particularly preferred aspects, the organic alcohol is ethanol. In some aspects, a mixture of organic alcohols can be used. If a mixture of organic alcohols are used, the plurality of organic alcohols in the mixture can be added simultaneously or in a staggered fashion. If the plurality of organic alcohols are added in a staggered fashion, a second alcohol can be added to a composition that already includes a first organic alcohol or excess first organic alcohol can be removed before the second organic alcohol is added. The organic alcohol can help to further solubilize the malonate composition. In addition, in some aspects, the presence of the organic alcohol in conjunction with the inorganic acid can also result in the esterification of at least a portion of the malonate composition. The amount of organic alcohol added is characterized using a molar concentration ratio of the organic alcohol:malonate composition. The molar concentration ratio of alcohol:malonate composition is typically in a range of 2:1 to 10:1 and preferably in a range of 4:1 to 8:1.

During acidification and solubilization, the malonate composition can be optionally heated (step 108). Typically, heating can occur at a temperature equal to or greater than 20° C. For example, heating can occur at a temperature in a range of 35° C. to 100° C., 40° C. to 90° C., or 40° C. to 80° C., or 40° C. to 70° C. Increasing the temperature can be helpful to solubilize the malonate composition and begin at least some degree of esterification of the malonate composition.

Acidification and solubilization of the malonate composition can occur for a desired amount of time. Acidification and solubilization typically occurs for at least 0.5 hours. Typical ranges of time for acidification and solubilization can be from 1 hour to 5 hours or 1 hour to 3 hours. Typically, acidification and solubilization ranges from 1 hour to 2 hours. Although acidification and solubilization can occur for longer amounts of time, an acceptable degree of acidification and solubilization can occur in the articulated ranges. The acidification and solubilization process can be deemed complete when the malonate composition is fully solubilized.

Acidification protonates formulas I and II. Protonation of formulas I and II, allows for a subsequent esterification reaction to occur using the protonated formulas I and II as well as any previously present free malonic acid as a reactant. This can allow for a higher yield of dialkyl malonate overall since more starting material is present. A particular benefit of this process is that dialkyl malonate compounds can be made without having to isolate any single component of the malonate composition for subsequent esterification. Separating any one component to be esterified adds unnecessary complexity and costs to the overall process. Also, the instant method can convert any of the malonate composition's components into a suitable reactant for esterification through the acidification. Additionally, the current method takes advantage of the fermentation method described herein and its ability to create a malonate composition that can be converted to a dialkyl malonate composition without the need to isolate a pure malonic acid stream. This is much easier overall than needing to create a new fermentation method that either creates the dialkyl malonate or selectively produces only one component of the malonate composition.

The acidification and solubilization also results in the formation of a precipitate of the M⁺ cation of formulas I and II and the conjugate base of the inorganic acid (e.g., SO₄²⁻ is the conjugate base of H₂SO₄). In various aspects, the precipitate is M₂SO₄, MHSO₄, MCl, MNO₃, M₃PO₄, M₂HPO₄, MH₂PO₄, or a mixture thereof. Typically, the precipitate is KHSO₄, K₂SO₄, or mixtures thereof.

In addition to the protonated form of formulas I and II, the presence of the inorganic acid and the organic alcohol will result in the esterification of at least some of the protonated form of formulas I and II as well as any free malonic acid that is present. The degree to which esterification occurs at this stage is controlled by at least two variables. Those variables are the amount of organic alcohol added during acidification and solubilization as well as the temperature that acidification and solubilization is carried out at. With respect to the amount of organic alcohol added, complete esterification can occur if an excess molar amount of the organic alcohol is added (i.e., more than 2 moles of alcohol per mole of malonic acid equivalents), and the water formed from the esterification reaction is substantially removed (i.e. 95% removed). Increasing the temperature during solubilization and acidification can also result in higher degrees of esterification, in addition to helping to solubilize the malonate composition.

Following acidification and solubilization, the produced acidified malonate composition includes an acidified malonate composition including a compound of formula IV:

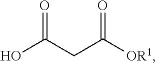

a compound of formula V:

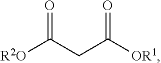

or
malonic acid (formula III), or a mixture thereof. In formulas IV and V, R¹ and R² are independently (C₁-C₈)alkyl. The precipitate of the M⁺ cation and conjugate base of the inorganic acid are also present. The amount of the compounds of formula IV and formula V as well as malonic acid depends on various factors including the pH of the solution and the amount of organic alcohol present. Upon equilibrium and a sufficient removal of water, if a large excess of organic alcohol is added as a reactant, the compound of formula V will be the most prevalent form. However, if only enough organic alcohol is added for solubilization or only a small excess of organic alcohol is added, then malonic acid is likely to be the prevalent form. If a small or moderate excess of organic alcohol is added, then the compound of formula IV is likely to be the prevalent form.

Typically, an excess molar amount of organic alcohol is added such that the compound of formula IV and the compound of formula V is at least 15 wt %, at least 30 wt % of the first acidified malonate composition. Preferably, if a large excess molar amount of organic alcohol is added, the compound of formula IV and the compound of formula V is at least 50 wt %, at least 60 wt % of the first acidified malonate composition.

The precipitate is removed (step 110) from the acidified malonate composition. Separating the precipitate from the acidified malonate composition can include filtering, centrifuging, settling, decanting, or a combination thereof. To help minimize product loss, the precipitate can be optionally rinsed with an organic alcohol following separation to recover any product that was attached or otherwise adhered to the precipitate. The organic alcohol used to wash the precipitate is typically the same organic alcohol that is used for acidification and esterification. In embodiments where heating step 108 occurs immediately following acidification and solubilization, a separation step 116 (which is analogous to step 110) occurs to separate the esterified malonate composition (which typically has at least 85 wt %, at least 90 wt %, at least 95 wt % of formula V) from the precipitate.

Separating the precipitate leaves the solution including the acidified malonate composition of formula IV, formula V, malonic acid, or a mixture thereof. Esterification at any carboxylic acid groups in the acidified malonate composition is then carried out (step 114) to form the dialkyl malonate ester. Esterification of the acidified malonate composition includes adding an organic alcohol to the acidified malonate composition. The organic alcohol is methanol, ethanol, propanol, butanol, pentanol, hexanol, heptanol, or octanol. Typically, the organic alcohol added is the same organic alcohol that was used for acidification and esterification. Esterification also includes heating the composition. The composition can be heated to a temperature of at least 50° C. Typically, the composition is heated to a temperature in a range of 50° C. to 110° C., 60° C. to 100° C., 65° C. to 85° C., or 65° C. to 75° C. Most preferably the temperature is not brought to or above 80° C. This is because at or above 80° C. malonic acid (as well as the compound of formula IV) has a tendency to decarboxylate. Heating occurs for an amount of time greater than or equal to 1 hour. For example, heating can occur from 1 hour to 20 hours or from 2 hours to 10 hours.

Esterification as described herein generally occurs according to the mechanism of a Fischer esterification. As generally understood, a Fischer esterification reaction produces water and an ester from the acid-catalyzed reaction between a carboxylic acid and an organic alcohol. The reaction is reversible. Therefore, production of the dialkyl malonate ester is driven forward, at least in part, by removing water during esterification. Prior to esterification, the malonate composition has a low water concentration. For example, a water concentration of the malonate composition is typically less than 5 wt % water and preferably, less than 4 wt %, 3 wt %, 2 wt %, 1 wt %, or less than 0.5 wt %.

To remove water that is formed, typically 1% to 30% of a total mass of liquid (excluding the dialkyl malonate ester) is removed per hour. Preferably, 1.5% to 18.5% or 1.0% to 15.3% of a total mass of liquid (excluding the dialkyl malonate ester) is removed per hour. The removed liquid includes water produced by esterification. The removed liquid can also include the organic alcohol. Organic alcohol that is removed, can optionally be recycled after separating the water from the organic alcohol, using any known process such as using a molecular sieve, and added back to the reaction to produce the dialkyl malonate ester. The liquid removed is in the form of an azeotrope of the organic alcohol and water. The amount of water removed, relative to the amount of ethanol that is removed and recycled, can be enhanced by adding a co-solvent to the azeotrope. The solvent is an organic solvent that is different from the organic alcohol used for esterification. Examples of suitable organic solvents include toluene, benzene, hexane, cyclohexane, heptane, xylene, ethylbenzene, or a mixture thereof. As an example, including toluene as a co-solvent with ethanol yields an azeotrope. This greatly enhances the rate at which water is removed and drives the esterification reaction forward. In an example, the azeotrope including toluene, ethanol, and water can include 12 wt % water, 37 wt % ethanol, and 51 wt % toluene. Following esterification, the reaction product present is 10 wt % to 99.9 wt % dialkyl malonate ester. Preferably, the reaction product present is 70% to 99.5% or from 90% to 99.5% dialkyl malonate ester. In addition to the dialkyl malonate ester reaction product, the composition can include leftover reactants, side products, or impurities. For example, the composition can include organic alcohols, inorganic acid, glycerol, arabitol, diethyl succinate, ethyl 3-hydroxypropanoate, ethyl 2,2-diethoxypropanoate, dextrose or other sugars, and excess water. Some of these are present in the original malonate composition and others can be formed during esterification.

The dialkyl malonate ester can be isolated from the leftover reactants, side products, or impurities mentioned above using distillation at step 118. Either the esterified malonate composition of step 114 or step 116 can be fed into the distillation process. If inorganic acid is present in the composition, it may be necessary to neutralize the inorganic acid prior to the distillation. The distillation process used is typically a fractional distillation process. Typically, distillation is performed at a temperature equal to or greater than 70° C. For example, distillation is performed at a temperature in a range of from 70° C. to 200° C., 70° C. to 180° C., 75° C. to 140° C., 80° C. to 130° C., or 90° C. to 100° C.

In addition to the temperature during distillation, the pressure during distillation is also controlled. Typically, the distillation occurs over two steps. During the first distillation step the pressure is typically in a range of from 16 mbar to 1,000 mbar, (for example, from 16 mbar to 1,000 mbar, from 26 mbar to 357 mbar, or 105 mbar to 138 mbar). During the first distillation, a temperature, in a first reboiler, is typically equal to or greater than 80° C. (for example, 80° C. to 201° C., 90° C. to 160° C., or 122° C. to 130° C.). During the second distillation step the pressure, measured at a top of a second distillation column, is typically from 1 mbar to 200 mbar, (for example, from 1 mbar to 200 mbar, from 2 mbar to 50 mbar, or 10 mbar to 19 mbar). Additionally, the second distillation step is conducted at a temperature, in a second reboiler, that is typically equal to or greater than 70° C. (for example, 70° C. to 196° C., 90° C. to 161° C., or 116° C. to 130° C.

In most aspects, the majority of any remaining water and ethanol are removed during the first distillation step. The diethyl malonate ester remains in the distillation bottoms. The distillate bottoms from the first distillation step are sent to the second distillation column. The diethyl malonate ester is then distilled from the other components in the distillation bottoms. In some alternative embodiments, the diethyl malonate ester along with low boiling components are taken off at the top in the first distillation column, and sent to the second distillation column. In that case, the low boiling components are then taken off the top in the second distillation column, while the diethyl malonate ester makes up the distillation bottoms.

The distillate produced by the distillation process described herein includes greater than 95 wt % dialkyl malonate ester, based on the total amount of liquid present in the distillate. For example, the distillate can include 95 wt % to 99.9 wt % dialkyl malonate ester based on the total amount of liquid present, 95 wt % to 99.5 wt % dialkyl malonate ester based on the total amount of liquid present, for example, at least 96 wt %, 97 wt %, 98 wt %, 99 wt %, or 99.9 wt % based on the total amount of liquid present. Preferably, the dialkyl malonate ester is diethyl malonate ester or dimethyl malonate ester. Even more preferably, the dialkyl malonate ester is diethyl malonate ester.

### EXAMPLES

Various embodiments of the present disclosure can be better understood by reference to the following Examples which are offered by way of illustration. The present disclosure is not limited to the Examples given herein. In the following examples, unless indicated to the contrary, X % means X wt %.

As used herein, references to a malonic acid (MA) mean a compound of formula III:

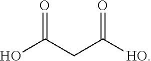

As used herein, references to a monosalt of malonic acid mean a compound of formula VI:

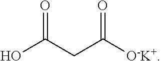

As used herein, references to a disalt of malonic acid mean a compound of formula VII:

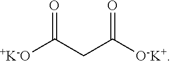

As used herein, references to a monoethyl malonate (MEM) ester mean a compound of formula VIII:

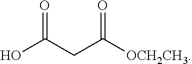

As used herein, references to a diethyl malonate ester (DEM) mean a compound of formula IX;

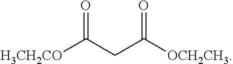

As used herein, the “DEM yield” of any yield of DEM discussed mean the yield percent as (moles of DEM/moles of malonic acid used in the reaction)*100.

In the Examples described herein the following analytical methods 1-4 are used to study reaction products formed from the procedures of the instant Examples.

### Analytical Method #1—GC Analysis of Diethyl Malonate Ester in Reaction Products

A gas chromatograph (GC), equipped with a flame ionization detector (FID), is used to separate and quantify diethyl malonate ester (DEM) from other reaction products that are formed in the reaction of malonic acid with ethanol in the presence of an acid catalyst. The gas chromatograph is an Agilent 6850 model, available from Agilent Technologies, Santa Clara, CA, equipped with a split/splitless injector (pulsed split ratio 25:1), set in split mode, with auto injection and a flame ionization detector (FID) detector. The column of the gas chromatograph is an Agilent DB-624, available from Agilent Technologies, Santa Clara, CA (30 m (length)×0.25 mm (diameter), 1.4 μm (particle size in the stationary phase)). The column temperature is initially set at 40° C. and held for 1 min, then ramped up to 60° C. at the rate of 8° C./min, then ramped up to 140° C. at 40° C./min, and finally ramped up to 200° C. at 7° C./min. The column temperature is then held at 200° C. for 1 min. The carrier gas hydrogen is supplied, as a carrier gas to the column, from a Parker Balston Hydrogen Generator, available from Parker Manufacturing Ltd, Gateshead UK. The injector temperature is 250° C., and the detector temperature is 250° C. Flow rate through the column is 2.5 mL/min-constant flow.

Calibration curves for both diethyl malonate ester and ethyl acetate are generated using standard solutions of commercially available pure compounds that were subsequently dissolved in acetonitrile. For analysis of the reaction mixtures, to which this method is applied, 100 microliters of the reaction mixture was mixed with 900 microliters of acetonitrile in a closed vial. The solution is thoroughly mixed, and 50 microliters of the solution are transferred to a fresh vial and diluted with 950 microliters of acetonitrile. After mixing thoroughly, 1 microliter of this solution is injected into the gas chromatograph.

### Analytical Method #2—High Pressure Ion Chromatographic System Analysis of Malonic Acid and Monoethyl Malonate Ester in Reaction Products

A high pressure ion chromatographic system (HPIC) is used to separate and quantify malonic acid (reactant) and monoethyl malonate ester (intermediate) formed in the esterification reaction of malonic acid and ethanol with a sulfuric acid catalyst. The HPIC is an ICS-4000 capillary ion chromatography system, available from Fischer Scientific, Waltham MA, that is equipped with an eluent generator, suppressor, and conductivity detector. The column is a Thermo Scientific IonPac AS 11-HC-4 μm (0.4×250 mm) capillary column, available from Thermo Fischer Scientific, Waltham MA. The flow rate through the column is 15 μL/min with a total run time of 60 minutes, at a column temperature of 30° C.

Analytes are separated and eluted off the column using a linear potassium hydroxide (KOH) concentration gradient ranging from 5 mM to 120 mM KOH (the gradient is linear from 0 to 31 minutes, this is followed by a 5 minute hold at 120 mM KOH, followed by a 24 minute equilibration at the starting conditions, 5 mM KOH). Calibration curves for both malonic acid and monoethyl malonate ester were generated using standard solutions of pure compounds in water. Analysis of a reaction mixture, to which this method is applied, is performed by mixing 100 microliters of the reaction mixture with 900 microliters of water in a closed vial. The obtained solution is thoroughly mixed, and 100 microliters are transferred to a fresh vial and diluted with another 900 microliters of water. After mixing thoroughly, 0.4 microliter of this solution is injected into the high pressure ion chromatographic system.

### Analytical Method #3—High Performance Liquid Chromatography Analysis of Malonic Acid

The high performance liquid chromatography method utilizes a guard column (Bio-Rad Cation H+ cartridge (p/n 125-0129) and holder (p/n 125-0131), both available from Bio-Rad Laboratories Hercules, CA); and two analytical columns in series (each of the two columns being Bio-Rad Aminex HPX-87H 300 mm×7.8 mm columns (125-0140), available from Bio-Rad Laboratories Hercules CA) in series. The injection volume for all runs is 10 μL. The mobile phase is isocratic (30 mM H₂SO₄). Each column temperature is held at 55° C. during each chromatography run. The flow rate through the columns is maintained at 0.6 mL/min, with each run duration being 45 minutes. Two detectors are used, namely, a refractive index detector (RID) (cell temperature held at 40° C.) and an ultraviolet detector (UVD) with the detector wavelength set at 210 nm.

### Analytical Method #4—Gas Chromatography Analysis of Monoethyl Malonate Ester and Diethyl Malonate Ester

The gas chromatography method utilizes an Agilent DB-17 column (30 m×0.25 mm OD, 0.5 μm film thickness (p/n 122-1733), available from Agilent Technologies, Santa Clara, CA. The injection volume is 1 μL with a split ratio of 100:1 and an injection port temperature of 275° C. A flow rate of 2 mL/min of helium is used. The temperature program has an initial setting of 50° C. and is held for 6 minutes. The temperature is increased to 250° C. at a rate of 25° C./minute and is held at that temperature for 6 minutes for a total run time of 20 minutes. A flame ionization detector is set at 275° C. with gas flow rates as follows—hydrogen: 35 mL/min, air: 350 mL/min, and makeup gas (nitrogen): 30 mL/min.

### Example 1

A multi-reactor X-plorer HT Parallel Bioprocessing unit PBL4L-2 poly-BLOCK, manufactured by HEL, Hertfordshire UK, is used for this example. Each reactor is set up with an agitator, a thermocouple to control the temperature, a short air-cooled condenser for venting the reaction, a port for introducing the acid catalyst, and a port for withdrawing samples. The port to withdraw samples has a three-way valve with 5 mL syringes attached to take samples (˜2 mL each time) during the course of the reaction.

Reagents used in Example 1 include:
Malonic acid, 99+% from Sigma-Aldrich, St. Louis MO
Anhydrous Ethanol, 99.5% (200 proof) from ACROS, Waltham MA
Concentrated (conc.) Sulfuric acid, 99.99% from Aldrich, St. Louis MO

### Example 1a—Effect of Temperature on the Reaction of Malonic Acid with Ethanol

The esterification of malonic acid with ethanol and a sulfuric acid catalyst is studied across a range of temperatures. In this Example, 70 g of malonic acid and 103.8 g of anhydrous ethanol are placed in each of 6 reactors. Separately, 4 g of conc. sulfuric acid is dissolved in 160 g of anhydrous ethanol at room temperature (e.g., 21° C.). The agitators are started (200 rpm), and the reactors are allowed to heat up to the desired temperatures. The reactions are run in duplicate, with temperatures set at 50° C., 60° C., and 70° C. When the desired temperatures are reached, 20.5 g of the above ethanol+sulfuric acid mixture is added to each reactor. The total amount of ethanol and sulfuric acid in the reactions, respectively, are shown below in Table 1. The addition of the sulfuric acid+ethanol solutions to the reactors is staggered every two minutes to allow for sample collection at the desired timepoints. The molar ratio of malonic acid:ethanol in the reactions is 1:4, the catalyst concentration is 0.69% (w/w) relative to malonic acid, and 0.25% (w/w) relative to the total mass of reactants.

TABLE 1

| | Malonic | | Sulfuric | | |
|---|---|---|---|---|---|
| Reactor # | Acid (g) | Ethanol (g) | Acid (g) | Total (g) | Temp (° C.) |
| 1 | 70 | 123.8 | 0.48 | 194.33 | 50 |
| 2 | 70 | 123.8 | 0.48 | 194.33 | 60 |
| 3 | 70 | 123.8 | 0.48 | 194.33 | 70 |
| 4 | 70 | 123.8 | 0.48 | 194.33 | 60 |
| 5 | 70 | 123.8 | 0.48 | 194.33 | 70 |
| 6 | 70 | 123.8 | 0.48 | 194.33 | 50 |

A 2 mL sample is withdrawn from each of the six reactors and transferred to vials soon after addition of the ethanol+sulfuric mixture. Following the transfer, the vials are immediately placed in dry ice. Subsequent samples are then taken from each of the reactors at 20, 40, 60, 90, 120, 180 240, 420, and 600 min and also placed in dry ice. The samples are analyzed by analytical methods #1 and #2, described herein. Table 2 shows the yields from each reaction at 600 min.

TABLE 2

| Reactor # | Temp (° C.) | MEM yield (%) | DEM yield (%) |
|---|---|---|---|
| 1 | 50 | 59 | 24 |
| 2 | 60 | 55 | 39 |
| 3 | 70 | 47 | 54 |
| 4 | 60 | 51 | 40 |
| 5 | 70 | 45 | 53 |
| 6 | 50 | 45 | 20 |

This example shows that the yield of diethyl malonate ester is greatest at 70° C., with decreasing yields at 60° C. and 50° C.

### Example 1b—Effect of Catalyst Concentration on the Reaction of Malonic Acid with Ethanol

The effect of sulfuric acid as a catalyst on the esterification of malonic acid is described herein. To study this, three solutions of concentrated sulfuric acid in anhydrous ethanol (H₂SO₄/EtOH) are prepared as follows:
Solution A—0.5 g concentrated sulfuric acid+60 g ethanol
Solution B—1.0 g concentrated sulfuric acid+60 g ethanol
Solution C—2.0 g concentrated sulfuric acid+60 g ethanol

70 g of malonic acid and 165 g of anhydrous ethanol are placed in each of 6 reactors. The agitators that are in contact with the 6 reactors and are started (200 rpm), and the reactors are allowed to heat up to 70° C. When the reactors reach 70° C., 20 g of Solution A is added to each of two reactors. Similarly, the same amounts of Solution B is added to two reactors, and the same amounts of Solution C is added to the remaining two reactors. The addition to each reactor is staggered every two minutes to facilitate sample collection at the desired timepoints. The molar ratio of malonic acid:ethanol in the reactions is 1:6, and the sulfuric acid catalyst concentrations are 0.24, 0.47, and 0.92% (w/w) relative to malonic acid. Results are shown in Table 3.

TABLE 3

| | | Malonic | | H₂SO₄/ | H₂SO₄ | | |
|---|---|---|---|---|---|---|---|
| Reactor | Solu- | Acid | Ethanol | EtOH | Catalyst | Total | Temp |
| # | tion | (g) | (g) | (g) | conc. (%) | (g) | (° C.) |
| 1 | A | 70 | 165.0 | 20.0 | 0.24 | 255.0 | 70 |
| 2 | B | 70 | 165.0 | 20.0 | 0.47 | 255.0 | 70 |
| 3 | C | 70 | 165.0 | 20.0 | 0.92 | 255.0 | 70 |
| 4 | A | 70 | 165.0 | 20.0 | 0.24 | 255.0 | 70 |
| 5 | B | 70 | 165.0 | 20.0 | 0.47 | 255.0 | 70 |
| 6 | C | 70 | 165.0 | 20.0 | 0.92 | 255.0 | 70 |

A 2 mL sample is withdrawn from each reactor and individually transferred to one of the six vials soon after addition of the respective H₂SO₄/EtOH mixture. Following the transfer, the vials are immediately placed in dry ice. Subsequent samples are then taken from each of the six reactors at 20, 40, 60, 90, 120, 180, 240, 420, and 600 min and also placed in dry ice. The samples are analyzed by Liquid Chromatography and Ion Chromatography. Table 4 below shows the yields from each reaction at 600 min.

TABLE 4

| Reactor # | Catalyst conc. (%) | DEM yield (%) |
|---|---|---|
| 1 | 0.24 | 46.44 |
| 2 | 0.47 | 53.32 |
| 3 | 0.92 | 65.49 |
| 4 | 0.24 | 45.53 |
| 5 | 0.47 | 45.84 |
| 6 | 0.92 | 64.10 |

The results showed that the highest DEM yield is obtained with a catalyst concentration of 0.92%. That result showed a maximum DEM yield of 64-65%.

### Example 1c—Effect of Malonic Acid to Ethanol Ratio on the Reaction

50 g of malonic acid (MA) and the ethanol quantities indicated in the table below are placed in six reactors, identified below in Table 5. Separately, 4.71 g of concentrated sulfuric acid is dissolved in 100 g of anhydrous ethanol at room temperature. The agitators are started (200 rpm), and all the reactors allowed to heat up to 70° C. When the reactors reach 70° C., 10 g of an ethanol and sulfuric acid mixture (abbreviated H₂SO₄/EtOH) is added to each reactor. The addition of the sulfuric acid and ethanol mixture is staggered every two minutes to allow for sample collection at the desired timepoints. The catalyst concentration is 0.9% (w/w) relative to Malonic acid.

TABLE 5

| | Malonic | | H₂SO₄/ | | | |
|---|---|---|---|---|---|---|
| Reactor | Acid | Ethanol | EtOH | Total | Temp | MA:EtOH |
| # | (g) | (g) | (g) | (g) | (° C.) | molar ratio |
| 1 | 50 | 122.7 | 10 | 182.69 | 70 | 1:6 |
| 2 | 50 | 166.9 | 10 | 226.92 | 70 | 1:8 |
| 3 | 50 | 211.2 | 10 | 271.15 | 70 | 1:10 |
| 4 | 50 | 122.7 | 10 | 182.69 | 70 | 1:6 |
| 5 | 50 | 166.9 | 10 | 226.92 | 70 | 1:8 |
| 6 | 50 | 211.2 | 10 | 271.15 | 70 | 1:10 |

A 2 mL sample is withdrawn from each reactor and transferred to vials soon after addition of the ethanol and sulfuric mixture, and the vials are immediately placed in dry ice. Subsequent samples are then taken from each reactor at 20 min, 40, 60, 90, 120, 180, 240, 420, and 600 min and also placed in dry ice. The samples are analyzed by Liquid Chromatography and Ion Chromatography. Table 6, below shows the yields from each reaction at 600 min.

TABLE 6

| Reactor # | MA:EtOH ratio | DEM yield (%) |
|---|---|---|
| 1 | 1:6 | 63.82 |
| 2 | 1:8 | 66.75 |
| 3 | 1:10 | 61.16 |
| 4 | 1:6 | 55.68 |
| 5 | 1:8 | 62.07 |
| 6 | 1:10 | 59.19 |

As demonstrated in Table 6, above, the highest DEM yield is in the reactions that are performed with a malonic acid:ethanol ratio (MA:EtOH ratio) of 1:8.

### Example 2

Example 2 shows that the conversion of malonic acid to DEM performed at a larger scale than demonstrated in Example 1.

Example 2 uses a 5 L glass reactor equipped with a heating jacket, an agitator, a thermocouple for temperature control, a glass condenser (for condensing vapors in reflux operation, or configured for distillation to remove water as an ethanol-water azeotrope), a port for introducing the acid catalyst and withdrawing samples. Chemicals used in Example 2 include:
Malonic acid 99%, available from Sigma-Aldrich, St. Louis MO
Ethanol 200 proof, available from Brenntag, Reading PA
Sulfuric acid 95.0-98.0%, available from Sigma-Aldrich, St. Louis MO

### Example 2a—Effect of Temperature and Molar Ratio on the Reaction of Malonic Acid with Ethanol

Ethanol is added to the reactor and the heating jacket is utilized to bring it up to the reaction temperature indicated in Table 7, below. Malonic acid is added during heating, and the mixture is allowed to reach the reaction temperature before addition of the sulfuric acid catalyst. The sulfuric acid catalyst concentration is 1% (w/w relative to malonic acid). The reaction mixtures are heated for the number of hours indicated in Table 7 below.

TABLE 7

| | Reac- | | | | | Sul- | |
|---|---|---|---|---|---|---|---|
| | tion | | Molar Ratio | Eth- | Malonic | furic | DEM |
| Run | Temp | Time | Ethanol:Malonic | anol | acid | acid | Yield |
| No. | (° C.) | (hrs) | acid | (g) | (g) | (g) | (%) |
| 1 | 70 | 17 | 4:1 | 1842.8 | 1040.6 | 10.41 | 66.8 |
| 2 | 50 | 19 | 4:1 | 1842.8 | 1040.6 | 10.41 | 59.3 |
| 3 | 70 | 21 | 10:1 | 2303.5 | 520.3 | 5.20 | 75.5 |
| 4 | 50 | 21 | 10:1 | 2303.5 | 520.3 | 5.20 | 47.7 |

The DEM content in the products are analyzed using Analytical Method #1. At both ethanol:malonic acid ratios tested, the DEM yield at a reaction temperature of 70° C. is greater than the DEM yield at a reaction temperature of 50° C.

### Example 2b—Effect of Continuously Removing Water in the Reaction of Malonic Acid with Ethanol

Ethanol is added to the reactor and the heating jacket utilized to bring it up to the reaction temperature specified herein at Table 8. Malonic acid is added during heating to the reaction temperature, and the mixture of malonic acid and ethanol is allowed to reach the desired temperature before addition of the sulfuric acid catalyst. The catalyst concentration is 1% (w/w relative to malonic acid).

When the equipment is configured for reflux operation (as in Example 2A), the glass condenser is placed vertically with all the condensed liquids re-entering the reactor. In runs 5 and 6, in Table 8 below, the condenser is oriented horizontally with all the condensed liquids collecting in a receiving flask. The reaction is initially run for 6 hrs at a reaction temperature of 70° C., while continuously removing the condensed liquid formed in the reaction as a water-ethanol azeotrope, by reducing the pressure to 30-50 kPa. After each hour of collecting the distillate, the vacuum is broken and a volume of 200 proof ethanol, equivalent in volume to the amount of distillate collected in the receiving flask, is added to the reactor, and the conditions are set back to a reaction temperature of 70° C. and 30-50 kPa. After the initial 6 hrs, the reaction is allowed to continue in a reflux mode overnight, at a reaction temperature of 70° C. without applying a vacuum. The DEM yield reported is at the end of this period is presented below in Table 9.

TABLE 8

| | | Initial | Molar Ratio | Initial | Malonic | Sulfuric |
|---|---|---|---|---|---|---|
| Run | Temp | reaction | Ethanol:Malonic | Ethanol | acid | acid |
| No. | (° C.) | time (hrs) | acid | (g) | (g) | (g) |
| 1 | 70 | 17 | 4:1 | 1842.8 | 1040.6 | 10.41 |
| 5 | 70 | 16 | 4:1 | 1842.8 | 1040.6 | 10.41 |
| 3 | 70 | 21 | 10:1 | 2303.5 | 520.3 | 5.20 |
| 6 | 70 | 17 | 10:1 | 2303.5 | 520.3 | 5.20 |

TABLE 9

| | Additional | | | |
|---|---|---|---|---|
| | Ethanol | Distillation | Distillation | DEM |
| Run No. | used (g) | pressure (kPa) | Time (hrs) | Yield |
| 1 | — | — | 0 | 66.8% |
| 5 | 5,000 | 30-50 | 6 | 94.2% |
| 3 | — | — | 0 | 75.5% |
| 6 | 5,000 | 30-50 | 6 | 88.6% |

The DEM content in the product is analyzed using Analytical Method #1. Distillation of water as an ethanol-water azeotrope allows the reaction to proceed to higher yields of DEM.

Examples 1 and 2 provide data relating to the conversion of pure malonic acid to DEM. Examples 3-5, however, provide data relating to the production of DEM from a fermentation broth that includes a malonate composition. As understood herein, a malonate composition can include malonic acid, monosalts of malonic acid, disalts of malonic acid, mixtures thereof, and other components as detailed herein. The fermentation broth can be associated with the fermentation process described herein above, and additional details on the fermentation process can be found in U.S. Provisional Patent Application No. 63/032,034, filed on May 29, 2020, the contents of which are hereby incorporated by reference.

### Example 3

The following example demonstrates the conversion of a malonate composition (primarily in the monosalt form) into malonic acid in the absence or presence of trace amounts of water. The malonate composition is precipitated or crystalized from a solution such as a fermentation broth. The absence of large amounts of water allows for the efficient precipitation and removal of the inorganic salt when the malonate composition is acidified with an inorganic acid, and helps the subsequent esterification reaction of malonic acid to proceed rapidly, while reducing the amount of water that has to be removed during the esterification reaction. Reducing the amount of water that has to be removed during esterification, also reduces the propensity for malonic acid degradation. This is because malonic acid tends to degrade at temperatures greater than 70° C., and especially at temperatures greater than 80° C., especially so in the presence of water.

Malonate compositions isolated from an aqueous solution are a mixture of free acid, monosalt, and disalt with the ratio driven by the pH of the solution. The composition of malonates isolated from an aqueous solution at pH 3 is 27 mol % malonic acid, 72 mol % monosalt of malonic acid, and 1 mol % disalt of malonic acid. The composition of malonate compositions isolated from an aqueous solution at pH 4 is 3 mol % malonic acid, 89 mol % monosalt of malonic acid, and 8 mol % disalt of malonic acid. A malonate composition isolated from a fermentation broth contains, in addition to the malonate compositions mentioned above, various other organic and inorganic compounds, including metals. A malonate composition isolated from a fermentation broth is described herein at Example 5.

### Example 3a—Acidification of Malonate Composition with Sulfuric Acid in Ethanol and Conversion to Diethyl Malonate Ester

To a one liter reaction flask equipped with a multi-neck lid and impeller, is added a malonate composition (85.8 g, 0.70 moles malonic acid equivalent basis) and anhydrous ethanol (228.9 g, 4.97 mol). The malonate composition is isolated from a pH 3 solution and comprises of 85 wt % malonic acid equivalents (27 mol % malonic acid, 72 mol % monosalt of malonic acid, and 1 mol % disalt of malonic acid), 0.4 wt % succinate, 0.3 wt % glucose, and 0.2 wt % 3-hydroxypropionate with most of the rest being potassium ions. The agitation is ramped to 250 rpm. Concentrated sulfuric acid (25.6 g, 0.25 mol) is added over a period of several minutes. Addition of sulfuric acid is at a rate to maintain the temperature below 30° C. Once sulfuric acid addition is complete, the reaction temperature (indicated in Table 10) is set to be reached as quickly as possible. The reaction is heated for a predetermined amount of time. Once the designated reaction time is reached, the reaction mixture is filtered and the resultant cake is rinsed with 60 mL ethanol.

The mass of filtrate, rinse, and cake (before and after drying) are recorded. The filtrate, rinse, and dried cake are analyzed according to analytical method #3 to determine malonic acid concentration, and the filtrate and rinse are also analyzed by analytical method #4 to determine the concentrations of diethyl malonate ester and monoethyl malonate ester. The phrase “amount of malonate recovered” as used in Table 10 means the % malonic acid equivalents in the malonate composition from the malonate composition that has been converted into malonic acid, monoethyl malonate (MEM), or diethyl malonate (DEM).

TABLE 10
Results of acidification of malonate composition

| | | | Amount of | Amount of |
|---|---|---|---|---|
| | | | malonic acid | malonic acid |
| | Reaction | Reaction | equivalents | equivalents |
| | time | temperature | recovered in | recovered in |
| Reaction | (hours) | (° C.) | the filtrate | the cake |
| 1 | 2 | 25 | 95.8% | 1% |
| 2 | 2 | 25 | 94.5% | 3% |
| 3 | 1 | 25 | 87.3% | 9% |
| 4 | 2 | 40 | 96.1% | 3% |
| 5 | 4 | 40 | 95.0% | 5% |
| 6 | 2 | 8 | 95.3% | 5% |

The filtrate is allowed to sit at room temperature for two weeks, and the malonic acid allowed to convert to the esters. At equilibrium, after two weeks, the malonic acid concentration is 2-5 mol % of the malonic acid equivalents in the acidified malonate composition, and the majority of the product is DEM. This is shown below at Table 11.

TABLE 11
Composition after equilibration

| | | | Equilibrium | Equilibrium |
|---|---|---|---|---|
| | | | diethyl | monoethyl |
| | Initial | Equilibrium | malonate | malonate |
| | malonic acid | malonic acid | ester in | ester in |
| | in filtrate | in filtrate | filtrate | filtrate |
| Reaction | (mol %)* | (mol %)* | (mol %)* | (mol %)* |
| 1 | 76.7% | 3.3% | 63.4% | 29.1% |
| 2 | 70.9% | 2.6% | 67.5% | 24.5% |
| 3 | 67.1% | 2.9% | 59.0% | 25.5% |
| 4 | 48.6% | 2.8% | 67.8% | 25.5% |
| 5 | 26.8% | 4.1% | 57.8% | 33.1% |
| 6 | 79.9% | 2.3% | 71.0% | 22.0% |

*mol % of the total malonic acid equivalents in the acidified malonate composition

To a one liter reaction flask equipped with a multi-neck lid and impeller, is added acetone (228.8 g, 3.94 moles) and a malonate composition (85.8 g, 0.70 moles) isolated from a pH 3 composition comprising 85 wt % malonate composition, 0.4 wt % succinate, 0.3 wt % glucose, and 0.2 wt % 3-hydroxypropionate. The agitation is ramped to 250 rpm. Concentrated sulfuric acid (25.6 g, 0.25 moles) is added over a period of several minutes at a rate to control reaction temperature below 30° C. Once sulfuric acid addition is complete, the reaction temperature is maintained at 25° C. for one hour. Once the reaction is complete, the reaction is filtered and the resultant cake is rinsed. The mass of filtrate, rinse, and cake (before and after drying) are recorded. The filtrate, rinse, and dried cake are analyzed by HPLC to determine malonic acid concentration. The average malonic acid recovery in the combined filtrate and rinse is 57.7%. An average of 32.8% malonate composition remains in the cake.

The above process is used when the goal is to isolate free malonic acid from a malonate composition (where the malonic acid is primarily in the monosalt form), using a non-reactive solvent. Other non-reactive solvents such as tetrahydrofuran or dioxane can be used. As seen above, the acidulation was allowed to proceed for only about 1 hour. Extending this time until complete acidulation will increase the yields.

Secondly, this process also allows for the re-crystallization of malonic acid from a non-reactive organic solvent. Using a solvent in which malonic acid is not highly soluble will allow higher recoveries of malonic acid during re-crystallization. Recrystallizing malonic acid from water requires several rounds of concentration and crystallization to maximize recovery due to the high solubility of malonic acid in water (which is greater than 60% at 25° C.).

### Example 4

Isolation of Malonate Compositions from Fermentation Broth

A fermentation broth comprising a malonate composition at pH 4 is clarified using microfiltration via a 100 nm ceramic membrane to remove cellular debris and other insoluble material. The clarified broth is concentrated to 70% total solids content via rotary evaporation at 60° C. and a pressure of 60 mbar.

To a one-liter reaction flask equipped with a multi-neck lid, impeller and pH probe, is added the concentrated clarified fermentation broth containing malonate composition. Agitation is started by ramping up to 250 RPM over 3 seconds and the temperature is increased to 70° C., over a 5-minute period. The temperature is then decreased to 8° C. at a rate of 1° C./minute. Once a temperature of 8° C. is reached, the reaction is held at that temperature for 120 minutes. After 120 minutes of holding at the set temperature, the reaction mixture is filtered. The mass of the crystals/precipitate after filtration and the filtrate are both recorded. The crystals/precipitate are dried to constant weight in an oven at 40° C. and reweighed. Both the crystals/precipitate and the filtrate are analyzed by analytical method #3 to determine amount of malonate composition and other components that are present. Percent recovery is determined for the solid and mass balance is calculated. Recovery of 62% of malonic acid equivalents in crystals/precipitate is observed in one crystallization. The solids from the single crystallization includes 62.5 wt % malonate composition based on malonic acid equivalents, 3.5 wt % glycerol, 1.2 wt % arabitol, 1 wt % maltose, 0.4 wt % glucose, 0.5 wt % 3-hydroxypropionate, 0.3 wt % succinate, 0.04 wt % pyruvate, less than 1 wt % moisture and greater than 20 wt % potassium ions.

The filtrate is evaporated to 65 wt % total solids and crystallized, filtered, and analyzed using the same procedure as for the first crystallization. The percent recovery in this second crystallization is 15.4 mol % of malonic acid equivalents from the malonate composition in the initial fermentation broth. The filtrate of the second crop is evaporated to 65 wt % total solids and crystallized, filtered, and analyzed using the same procedure as the first crystallization. The percent recovery in this third crystallization is 9.2 mol % of malonic acid equivalents from the malonate composition in the initial fermentation broth. The overall recovery on a malonic acid equivalents basis for three compositions is 87.1%.

### Example 5

Acidification of Malonate Composition Isolated from a Fermentation Broth with Sulfuric Acid

To a one-liter reaction flask, equipped with a multi-neck lid and impeller, is added the malonate composition (100 g, 0.60 moles malonic acid equivalents basis) isolated from a malonic acid fermentation broth (described herein in example 4) and anhydrous ethanol (196.1 g, 4.9 mol). The agitation is ramped to 250 rpm. Concentrated sulfuric acid (59 g, 0.60 mol) is added over a period of several minutes at a rate to maintain the temperature below 40° C. Once the sulfuric acid addition is complete, the reaction temperature is maintained at 40° C. for 2 hrs. Once the designated reaction time is reached, the reaction is filtered and the resultant cake is rinsed with room temperature anhydrous ethanol. The mass of filtrate, rinse, and cake (before and after drying) are recorded. Greater than 95 mol % of the of the malonic acid equivalents of the malonate compositions present in the crystals/precipitate isolated in Example 4 is recovered as a mixture of malonic acid, monoethyl malonate, and diethyl malonate in the combined filtrate and rinse solution.

Acidification of Malonate Composition Isolated from a Fermentation Broth with Sulfuric Acid

To a 500 mL, 3-neck reaction flask in a heating mantle and with a magnetic stir bar and agitation set at 400 rpm is added a solution of concentrated sulfuric acid (92.1 g, 0.94 mol) in anhydrous ethanol (287 g, 6.23 mol). To this solution is added a malonate composition (100 g, 0.63 moles malonic acid equivalent basis). The malonate composition is isolated from a fermentation that is maintained at pH 4 via addition of potassium hydroxide and comprises 65 wt % malonate acid equivalents, 0.2 wt % succinate, 3 wt % glucose, 0.7 wt % maltose, 0.5 wt % arabitol, 2.7 wt % 3-hydroxypropionate, 0.1 wt % pyruvate, and 0.1 wt % glycerol with most of the rest being potassium ions. The reaction temperature is set at 70° C. and the reaction is continued for two hours. Aliquots from the reaction mixture are pulled at 0, 0.5, 1, and 2 hours for analysis. The reaction mixture is filtered via GF/A paper and the resultant cake is rinsed with 257 g ethanol. The filtrate and cake are analyzed by analytical method #3 to determine malonic acid concentration and GC analytical method #4 to determine the concentrations of diethyl malonate ester and monoethyl malonate ester. Malonate conversion refers to the malonic acid equivalents in the malonate composition that is converted to MEM and/or DEM. Results are shown in Table 12.

TABLE 12
Results of acidification and esterification of malonate composition

| Reaction | | | Malonate |
|---|---|---|---|
| Time (hrs) | DEM % yield | MEM % yield | Conversion (%) |
| 0 | 4.1% | 9.5% | 13.7% |
| 0.5 | 30.7% | 8.4% | 39.1% |
| 1 | 49.5% | 15.9% | 65.4% |
| 2 | 71.9% | 24.8% | 96.8% |

### Example 6

### Kinetic Properties

Aspen Custom Modeler, within the Aspen Plus suite of software, available from Aspentech, Bedford MA, is used to model the kinetic properties of the esterification of malonic acid to monoethyl malonate ester (MEM) and diethylmalonate (DEM). In experiments to generate data for the determination of kinetic parameters for the esterification of malonic acid to monoethyl malonate ester and diethyl malonate ester, batch reactors are dosed with ethanol, malonic acid, and sulfuric acid. Reaction conditions are varied, including varying the temperature from 50° C. to 70° C., ethanol to malonic acid molar ratios of 4:1 to 10:1, and sulfuric acid catalyst loadings of 0.57 to 3.41 g/L.

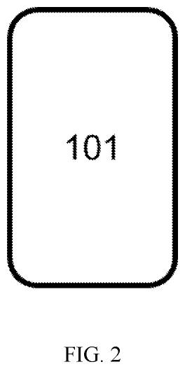

Fits of the experimental results for a molar ratio of ethanol to malonic acid of 4:1 is used with sulfuric acid loading of 1.7 g/L, shown in FIG. 2, generate the kinetic parameters, shown in Table 13a, to the Arrhenius equations below for the forward and reverse esterification reactions. E and W mean ethanol and water respectively.

$$MA + E {⇌}_{{k}_{- 1}}^{{k}_{1}} MEM + W$$

$$MEM + E {⇌}_{{k}_{- 2}}^{{k}_{2}} DEM + W$$

TABLE 13a
Kinetic Parameters

| | Eₐ | A |
|---|---|---|
| Reaction | (kJ mol⁻¹) | (M⁻¹ min⁻¹) |
| MA + E → MEM + W | 52.03 | 146,317 |
| MEM + W → MA + E | −8.81 | 0.00107 |
| MEM + E → DEM + W | 44.07 | 2,404 |
| DEM + W → MEM + E | −15.73 | 0.000184 |

### Example 6a: Batch Reaction

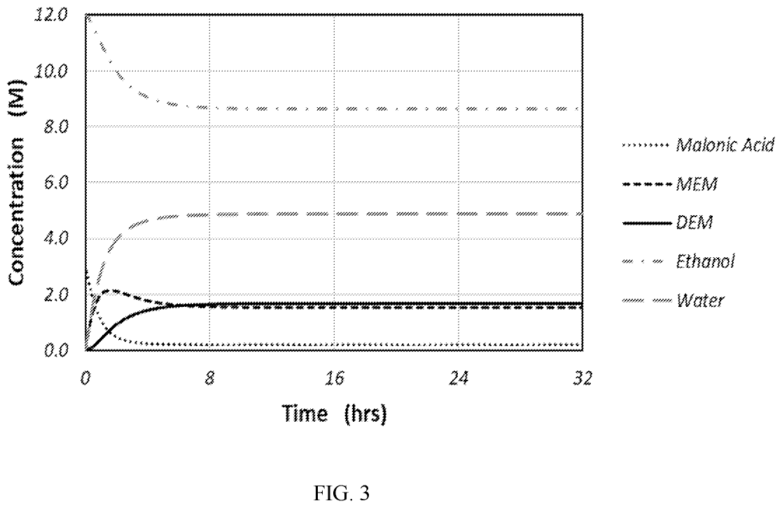

Examples are then generated from Aspen Custom Modeler for a batch reactor, FIG. 2. In each example, the batch reactor, 101, is dosed with a representative fermentation product from Example 4. The representative fermentation product includes malonic acid (89.9 wt %), glycerol (5.0 wt %), arabitol (1.7 wt %), 3-hydroxypropionic acid (0.7 wt %), succinic acid (0.4 wt %), pyruvic acid (0.1 wt %), dextrose (0.6 wt %), and higher sugars (1.4 wt %). The fermentation product is dosed with ethanol at a molar ratio relative to malonic acid of 4:1, sulfuric acid at 1.7 g/L, and held at 70° C. for the extent of the reaction, to match the experimental conditions used to measure the kinetic parameters. Kinetics were not measured for the other carboxylic acids present in the fermentation product solution, but given the acidic reaction environment with an excess of ethanol present full conversion to the esterified products is assumed. FIG. 3 shows the theoretical profile for this system, with an equilibrium approached in 10 hours. The results for the equilibrium conditions after 32 hours of reaction time are shown in Table 13b. Without water removal, a significant amount of the starting malonic acid is esterified, although almost half of the converted malonic acid remains as MEM, rather than fully esterifying to DEM.

TABLE 13b
Batch Reactor Results

| Temperature | 70° | C. |
|---|---|---|
| C_{MA} at t = 0 hrs | 3.02M | |
| C_{EtOH} at t = 0 hrs | 12.03M | |
| C_{MA} at t = 32 hrs | 0.19M | |
| C_{MEM} at t = 32 hrs | 1.52M | |
| C_{DEM} at t = 32 hrs | 1.67M | |
| C_{EtOH} at t = 32 hrs | 8.62M | |
| C_{Water} at t = 32 hrs | 4.87M | |
| MA Conversion | 94.3 | mol % |
| DEM Yield | 49.4 | mol % |

### Example 6b: Batch Reaction with Water Removal

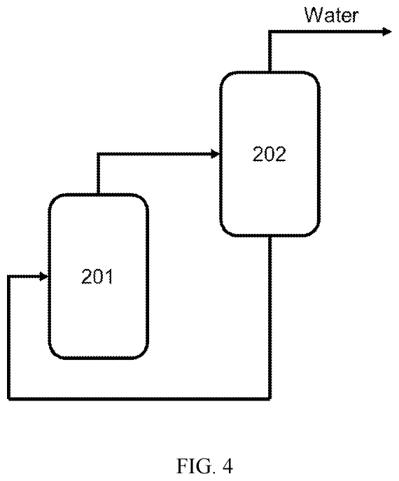

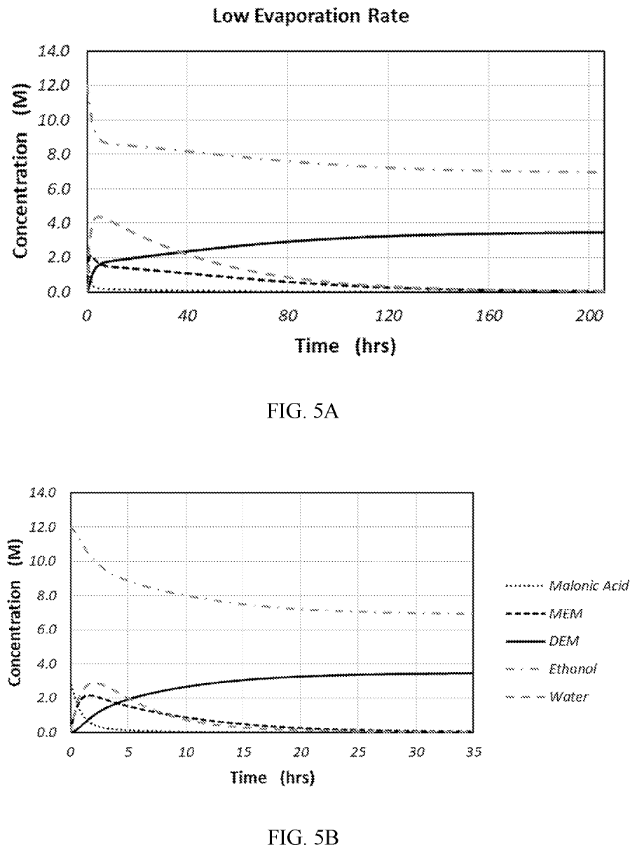

Examples are then generated from the Aspen Custom Modeler for a batch reactor with water removal, FIG. 4. In each example, the batch reactor, 201, is dosed with the representative fermentation product of Example 6a. The fermentation product is dosed with ethanol at a molar ratio relative to malonic acid of 4:1, sulfuric acid at 1.7 g/L, and held at 70° C. for the extent of the reaction, to match the experimental conditions used to measure the kinetic parameters. Kinetics were not measured for the other carboxylic acids present in the fermentation product solution, but given the acidic reaction environment with an excess of ethanol present full conversion to the esterified products is assumed. Flash vapors are taken off from the reactor at a pressure of 400 mbar at two different rates of removal; a low average evaporation rate of 1.5% of the initially dosed mass per hour and a high average evaporation rate of 15.3% of the initially dosed mass per hour. The evaporation rate at the held pressure and temperature of the reaction vessel is controlled by changing the rate of heat input into the system. Water is selectively removed from the flash vapors, 202, using molecular sieves or similar technology, while the rest of the reactants and products in the flash vapors are returned to the batch reactor in order to drive the equilibrium towards DEM. FIGS. 5A and 5B show the theoretical profiles for this systems, where the system is allowed to run until a 99 mol % yield of DEM is achieved. FIG. 5A shows the theoretical profile for the low average evaporation rate and FIG. 5B shows the theoretical profile for the high average evaporation rate. The results for these systems are shown in Table 13c.

Drawing vapors at a higher rate allows for a faster rate of conversion of malonic acid to DEM, with a 99% molar yield of DEM achieved in 35 hours rather than 206 hours. This is due to the faster removal of water from the reacting system, driving the equilibrium forward more rapidly. Those higher vapor rates, however, also result in a greater recycle rate of ethanol, possibly requiring larger unit operations to process and recycle those materials.

TABLE 13c
Batch Reactor Results with Water Removal

| Temperature | 70° | C. | 70° | C. |
|---|---|---|---|---|
| Flash Pressure | 400 | mbar | 400 | mbar |
| Average Flash Rate | Low average | | High average | |
| | evaporation rate (1.5% | | evaporation rate (15.3% | |
| | Initial Mass per Hour) | | Initial Mass per Hour) | |
| C_{MA} at t = 0 hrs | 3.02M | | 3.02M | |
| C_{EtOH} at t = 0 hrs | 12.03M | | 12.03M | |
| t_{Final} | 206 | hrs | 35 | hrs |
| C_{MA} at t_{Final} | 0.00M | | 0.00M | |
| C_{MEM} at t_{Final} | 0.04M | | 0.03M | |
| C_{DEM} at t_{Final} | 3.45M | | 3.45M | |
| C_{EtOH} at t_{Final} | 6.94M | | 6.95M | |
| C_{Water} at t_{Final} | 0.04M | | 0.01M | |
| Water Removed | 99.4% | | 99.8% | |
| Ethanol Recycled* | 557% | | 969% | |
| MA Conversion | 100.0% | | 100.0% | |
| DEM Yield | 99.0 | mol % | 99.0 | mol % |

*% relative to ethanol initially dosed in reactor

### Example 6c: Batch Reaction with Water Removal Using a Toluene Co-Solvent

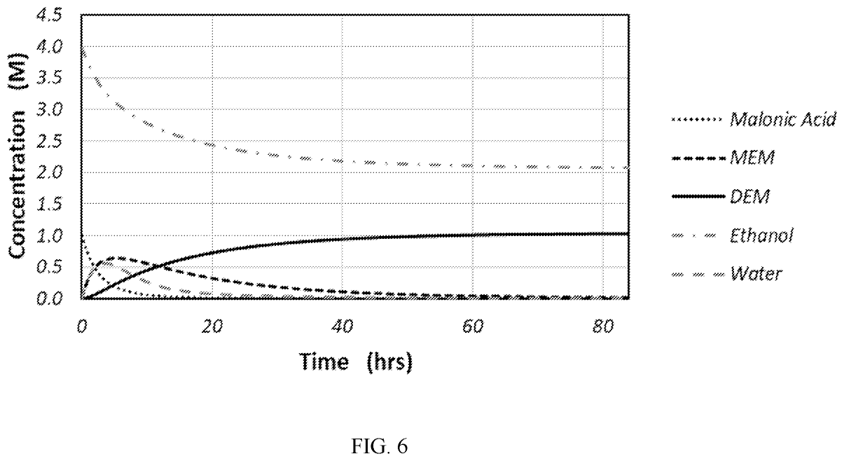

One solution to the issue of ethanol recycling involves using a co-solvent, such as toluene, to decrease the amount of ethanol that is evaporated from the system. Examples are generated from the Aspen Custom Modeler for a batch reactor with water removal using a toluene co-solvent. In each example, the batch reactor, 201 (shown in FIG. 4), is dosed with a representative malonic acid (89.9 wt %), glycerol (5.0 wt %), arabitol (1.7 wt %), 3-hydroxypropionic acid (0.7 wt %), succinic acid (0.4 wt %), pyruvic acid (0.1 wt %), dextrose (0.6 wt %), and higher sugars (1.4 wt %). The fermentation product is dosed with ethanol at a molar ratio relative to malonic acid of 4:1, sulfuric acid at 1.7 g/L, toluene at a mass ratio relative to ethanol of 3:1 and held at 70° C. for the extent of the reaction, to match the experimental conditions used to measure the kinetic parameters. Kinetics have not been measured for the other carboxylic acids present in the fermentation product solution, but given the acidic reaction environment with an excess of ethanol present full conversion to the esterified products is assumed. Flash vapors are taken off from the reactor at a pressure of 550 mbar at an average evaporation rate of 1.5% of the initially dosed mass per hour. Water is selectively removed from the flash vapors, 202, using molecular sieves or similar technology, while the rest of the reactants and products in the flash vapors are returned to the batch reactor in order to drive the equilibrium towards DEM. FIG. 6 shows the theoretical profiles for this systems, where the system is allowed to run until a 99 mol % yield of DEM is achieved. The results for these systems are shown in Table 13d.

For the same low evaporation rate from the system used in Example 6b, the time to reach 99 mol % yields of DEM decreases from 206 hours to 84 hours and the amount of ethanol that needs to be recycled back to the reactor decreases from 557% of the initial dose to 336%. In addition, the system can be run at a higher pressure, 550 mbar instead of 400 mbar.

TABLE 13d
Batch Reactor Results with Water
Removal Using Toluene Co-Solvent

| Temperature | 70° | C. |
|---|---|---|
| Flash Pressure | 550 | mbar |
| Average Flash Rate | Low average | |
| | evaporation rate (1.5% | |
| | Initial Mass per Hour) | |
| C_{MA} at t = 0 hrs | 1.00M | |
| C_{EtOH} at t = 0 hrs | 3.97M | |
| t_{Final} | 84 | hrs |
| C_{MA} at t_{Final} | 0.00M | |
| C_{MEM} at t_{Final} | 0.01M | |
| C_{DEM} at t_{Final} | 1.03M | |
| C_{EtOH} at t_{Final} | 2.08M | |
| C_{Water} at t_{Final} | 0.00M | |
| Water Removed | 99.9% | |
| Ethanol Recycled* | 336% | |
| MA Conversion | 100.0% | |
| DEM Yield | 99.0 | mol % |

*% relative to ethanol initially dosed in reactor

### Example 7

### Fractional Distillation of Esterification Product to Produce High Purity Diethyl Malonate Ester

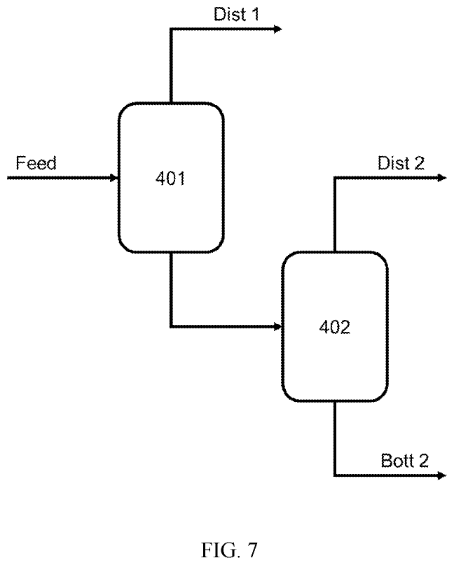

Using the representative fermentation product at the end of the esterification reaction in-Examples 6b, the following composition, malonic acid (0.00 wt %), MEM (0.49 wt %), DEM (60.10 wt %), ethanol (34.76 wt %), sulfuric acid (0.21 wt %), glycerol (2.21 wt %), arabitol (0.76 wt %), diethyl succinate (0.19 wt %), ethyl 3-hydroxypropionate (E-3HP, 0.32 wt %), ethyl 2,2-diethoxypropionate (0.03 wt %), dextrose (0.25 wt %), higher sugars (0.63 wt %), and water (0.03 wt %) is obtained. The sulfuric acid is first neutralized with sodium ethoxide and the sodium sulfate that forms is filtered off. The filtered product is then sent to the two-column, fractional distillation setup shown in FIG. 7. The product after esterification and neutralization is sent to the first distillation column, 401, with a top pressure of 200 mbar and temperature of 74.8° C. and bottom pressure of 248 mbar and temperature of 148.0° C., where the majority of the remaining ethanol and water are distilled out. The bottoms of the first distillation column are sent to a second distillation column, 402, with a top pressure of 50 mbar and temperature of 103.6° C. and bottom pressure of 98 mbar and temperature of 175.9° C., where DEM product is distilled from the heavy components. Table 4a shows the inlet and outlet stream flows and concentrations. As can be seen, 98.8% of the DEM product is recovered at a purity of 99.46%. As can be seen, all of the similar vapor pressure of ethyl 3-hydroxypropionate to DEM causes all of this fermentation side product to exit in the distillate with the DEM product, putting an upper limit on the final purity of 99.47%. Results are shown in Table 14

TABLE 14
Distillation Flows and Compositions

| | | Dist 1 | | Bott 2 | | Dist 2 | | |
|---|---|---|---|---|---|---|---|---|
| | Feed | | Percent | | Percent | | Percent | |
| | Flow | Flow | of Feed | Flow | of Feed | Flow | of Feed | Conc. |
| Component | (kg/hr) | (kg/hr) | (mass %) | (kg/hr) | (mass %) | (kg/hr) | (mass %) | (mass %) |
| DEM | 60.10 | 0.00 | 0.0% | 0.74 | 1.2% | 59.36 | 98.8% | 99.46% |
| MEM | 0.49 | 0.00 | 0.0% | 0.49 | 100.0% | 0.00 | 0.0% | 0.00% |
| Malonic Acid | 0.00 | 0.00 | 0.0% | 0.00 | 100.0% | 0.00 | 0.0% | 0.00% |
| Ethanol | 34.76 | 34.75 | 100.0% | 0.00 | 0.0% | 0.01 | 0.0% | 0.01% |
| Water | 0.03 | 0.03 | 99.9% | 0.00 | 0.1% | 0.00 | 0.0% | 0.00% |
| Dextrose | 0.25 | 0.00 | 0.0% | 0.25 | 100.0% | 0.00 | 0.0% | 0.00% |
| Higher Sugars | 0.63 | 0.00 | 0.0% | 0.63 | 100.0% | 0.00 | 0.0% | 0.00% |
| Glycerol | 2.21 | 0.00 | 0.0% | 2.21 | 100.0% | 0.00 | 0.0% | 0.00% |
| Arabitol | 0.76 | 0.00 | 0.0% | 0.76 | 100.0% | 0.00 | 0.0% | 0.00% |
| ME-Succ* | 0.03 | 0.00 | 0.0% | 0.03 | 100.0% | 0.00 | 0.0% | 0.00% |
| DE-Succ** | 0.19 | 0.00 | 0.0% | 0.19 | 100.0% | 0.00 | 0.0% | 0.00% |
| E-3HP*** | 0.32 | 0.00 | 0.0% | 0.00 | 0.0% | 0.32 | 100.0% | 0.53% |
| E-2,2DEProp # | 0.03 | 0.00 | 0.0% | 0.02 | 89.8% | 0.00 | 10.2% | 0.00% |

*Monoethyl succinate,
**Diethyl succinate,
***Ethyl 3-hydroxypropionate,
# Ethyl 2,2-diethoxypropionate.

The terms and expressions employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Additional Aspects.

The following exemplary aspects are provided, the numbering of which is not to be construed as designating levels of importance:

Aspect 1 provides a method for making a dialkyl malonate ester, the method comprising:
a) acidifying a malonate composition comprising:
at least 30 wt % based on malonic acid equivalents of a compound of formula I:

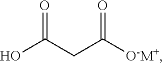

wherein M⁺ is a Group I alkali metal cation or ammonium cation, and wherein the malonate composition optionally further comprises at least one of:

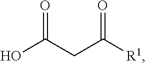

and
malonic acid,
wherein the acidifying of the malonate composition comprises contacting an organic alcohol and an inorganic acid with the malonate composition to solubilize the malonate composition to form a first acidified malonate composition comprising at least one of:
a compound of formula IV

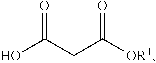

a compound of formula V

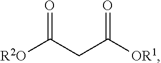

and
malonic acid,
wherein R¹ and R² are independently (C₁-C₈)alkyl, and
optionally heating the malonate composition, the first acidified malonate composition, or both, to form the dialkyl malonate ester;
b) separating a precipitate comprising M⁺ from the product of step a) to form a second acidified malonate composition comprising the compound of formula IV the compound of formula V, malonic acid, or a mixture thereof, and optionally heating the second acidified malonate composition to form the dialkyl malonate ester, wherein the optional heating of step a), step b), or both, is performed to form the dialkyl malonate ester;
c) distilling the product of step b) to isolate a distillate comprising the dialkyl malonate ester, wherein the dialkyl malonate ester is greater than or equal to 90 wt % of the distillate (for example, 90 wt % to 100 wt % or 95 wt % to 99.9 wt % or 95 wt % to 99.5 wt %).

Aspect 2 provides the method of Aspect 1, wherein acidifying the malonate composition of step a) is conducted using a 0.2:1.0 to 2.0:1.0 equivalents of protons in inorganic acid to equivalents of M⁺ (for example, 0.5:1.0 to 1.5:1.0, 0.7:1.0 to 1.2:1.0).

Aspect 3 provides the method of any one of Aspects 1 or 2, wherein during step a) a pH of the malonate composition is brought to a pH less than or equal to 2 (for example, a pH of 0.1 to 2, a pH of 0.1 to 1.5, or a pH of 0.1 to 1).

Aspect 4 provides the method of any one of Aspects 1-3, wherein during step a) the malonate composition is heated to a temperature equal to or greater than 20° C. (for example, in a range of 35° C. to 100° C., 40° C. to 90° C., or 40° C. to 80° C., 40° C. to 70° C.).

Aspect 5 provides the method of any one of Aspects 1-4, wherein the dialkyl malonate ester comprises a (C₁-C₈)dialkyl malonate ester.

Aspect 6 provides the method of Aspect 5, wherein (C₁-C₈)dialkyl malonate ester comprises a (C₁-C₆)dialkyl malonate ester.

Aspect 7 provides the method of Aspect 6, wherein (C₁-C₆)dialkyl malonate ester comprises a (C₁-C₄)dialkyl malonate ester (for example, a (C₁-C₃)dialkyl malonate ester or a (C₁-C₂)dialkyl malonate ester).

Aspect 8 provides the method of any one of Aspects 1-7 wherein the organic alcohol of step a) is methanol, ethanol, propanol(branched or linear), butanol(branched or linear), pentanol(branched, linear, or cyclic), hexanol(branched, linear, or cyclic), heptanol(branched or linear), or octanol(branched or linear) (for example, methanol or ethanol).

Aspect 9 provides the method of any one of Aspects 1-8, further comprising adding an organic alcohol (for example, methanol, ethanol, propanol (branched or linear), butanol(branched or linear), pentanol(branched, linear, or cyclic), hexanol(branched, linear, or cyclic), heptanol(branched or linear), or octanol(branched or linear)) during step b).

Aspect 10 provides the method of Aspect 9, wherein the organic alcohol added during step b) is the same organic alcohol (for example, methanol, ethanol, propanol (branched or linear), butanol(branched or linear), pentanol(branched, linear, or cyclic), hexanol(branched, linear, or cyclic), heptanol(branched or linear), or octanol(branched or linear)) added to the malonate composition during step a).

Aspect 11 provides the method of any one of Aspects 1-10, wherein a water concentration of the malonate composition, prior to acidification during step a) is less than 5 wt % (for example, less than 4 wt %, 3 wt %, 2 wt %, 1 wt %, or less than 0.5 wt %) prior to step b).

Aspect 12 provides the method of any one of Aspects 1-11, wherein during step b), the first acidified malonate composition of step a) or the second acidified malonate composition of step b) is heated to a temperature of at least 50° C. (for example, in a range of 50° C. to 110° C., 60° C. to 100° C., 65° C. to 85° C., or 65° C. to 75° C.).

Aspect 13 provides the method of any one of Aspects 1-12, wherein the second acidified malonate composition is heated during step b).

Aspect 14 provides the method of any one of Aspects 1-13, wherein during the distillation step c) a first distillation step is conducted at a temperature, in a first reboiler, equal to or greater than 80° C. (for example, 80° C. to 201° C., 90° C. to 160° C., or 122° C. to 130° C.) and a second distillation step is conducted at a temperature, in a second reboiler, equal to or greater than 70° C. (for example, 70° C. to 196° C., 90° C. to 161° C., or 116° C. to 130° C.

Aspect 15 provides the method of any one of Aspects 1-14, wherein during the distillation step c) a first distillation step is conducted at a pressure, measured at a top of a first distillation column, in a range of from 16 mbar to 1,000 mbar, (for example, from 16 mbar to 1,000 mbar, from 26 mbar to 357 mbar, or 105 mbar to 138 mbar) and a second distillation step is conducted at a pressure, measured at a top of a second distillation column of from 1 mbar to 200 mbar, (for example, from 1 mbar to 200 mbar, from 2 mbar to 50 mbar, or 10 mbar to 19 mbar).

Aspect 16 provides the method of any one of Aspects 14 or 15, wherein a first distillate from the first distillation step is enriched in ethanol and water, and distillate bottoms from the first distillation step is distilled to produce a second distillate enriched in dialkyl malonate ester from the second distillation step.

Aspect 17 provides the method of any one of Aspects 1-16, wherein the organic alcohol of step a) is methanol, ethanol, or a mixture thereof and the dialkyl malonate ester of step c) comprises dimethyl malonate ester or diethyl malonate ester, or a mixture thereof.

Aspect 18 provides the method of any one of Aspects 1-17, wherein the compound of formula I is present in the malonate composition of step a) in a greater amount by wt % than the compound of formula II.

Aspect 19 provides the method of any one of Aspects 1-18, wherein the malonate composition prior to acidification during step a) comprises: at least 50 wt % (for example, at least 55 wt %, at least 60 wt %) of the compound of formula I; no greater than 25 wt % (for example, no greater than 20 wt %, no greater than 15 wt %, no greater 10 wt %, or not greater 5 wt %) of the compound of formula II; and less than 50 wt % (less than 45 wt %, less than 40 wt %, less than 35 wt %, less than 30 wt %) malonic acid.

Aspect 20 provides the method of any one of Aspects 1-19, wherein the compound of formula IV and the compound of formula V comprises at least 15 wt %, at least 30 wt % of the first acidified malonate composition of step a) or the second acidified malonate composition of step b).

Aspect 21 provides the method of Aspect 20, wherein the compound of formula IV and the compound of formula V comprises at least 50 wt %, at least 60 wt % of the first acidified malonate composition of step a) or the second acidified malonate composition of step b).

Aspect 22 provides the method of any one of Aspects 1-21, wherein acidifying the first malonate composition during step a) is conducted for at least 0.5 hours (for example, in a range of 1 hour to 5 hours or 1 hour to 3 hours).

Aspect 23 provides the method of Aspect 22, wherein acidifying the malonate composition during step a) is conducted for a time in a range of 1 hour to 2 hours.

Aspect 24 provides the method of any one of Aspects 1-23, wherein the separating of the precipitate during step b) comprises subjecting the first acidified malonate composition of step a) to filtering, centrifuging, settling, decanting, or a combination thereof, and optionally further comprises rinsing the precipitate with the alcohol utilized during acidification during step a).

Aspect 25 provides the method of any one of Aspects 1-24, wherein M⁺ comprises Na⁺ or K⁺, or a combination thereof.

Aspect 26 provides the method of Aspect 25, wherein M⁺ is K⁺.

Aspect 27 provides the method of any one of Aspects 1-26, wherein the precipitate comprises M₂SO₄, MHSO₄, MCl, MNO₃, M₃PO₄, M₂HPO₄, MH₂PO₄, or a mixture thereof.

Aspect 28 provides the method of Aspect 27 wherein the inorganic acid comprises sulfuric acid, hydrochloric acid, nitric acid, phosphoric acid, or mixtures thereof.

Aspect 29 provides the method of any one of Aspects 27 or 28, wherein M comprises Na, K, or a mixture thereof and the inorganic acid is selected from sulfuric acid, hydrochloric acid, nitric acid, phosphoric acid, or mixtures thereof.

Aspect 30 provides the method of any one of Aspects 1-29, wherein the inorganic acid comprises sulfuric acid and the precipitate separated during step b) comprises KHSO₄, K₂SO₄, or mixtures thereof.

Aspect 31 provides the method of any one of Aspects 1-30, wherein during step a) the organic alcohol is added at a molar concentration ratio, relative to the malonic acid equivalents, in a range of 2:1 to 10:1 (EtOH:malonic acid equivalents).

Aspect 32 provides the method of Aspect 31, wherein the molar concentration ratio, relative to the malonic acid equivalents, is in a range of 4:1 to 8:1 (EtOH:malonic acid equivalents).

Aspect 33 provides the method of any one of Aspects 1-32, wherein the heating during step b) is performed at a temperature of at least 50° C. (for example, in a range of, 50° C. to 110° C., 60° C. to 100° C., 65° C. to 85° C., or 65° C. to 75° C.).

Aspect 34 provides the method of any one of Aspects 1-33, wherein the heating during step b) is performed for an amount of time greater than or equal to 1 hour (for example, from 1 hour to 20 hours, 2 hours to 10 hours).

Aspect 35 provides the method of Aspect 34, wherein the heating during step b) is performed for an amount of time in a range of from 2 hours to 10 hours.

Aspect 36 provides the method of any one of Aspects 1-35, wherein step c) further comprises removing water from the first acidified malonate composition of step a) or the second acidified malonate composition of step b).

Aspect 37 provides the method of any one of Aspects 1-36, wherein the compound of formula IV and formula V comprises 15 wt % to 99.9 wt % (for example, 70 wt % to 99 wt %) of the first acidified malonate composition of step a) or the second acidified malonate composition of step b).

Aspect 38 provides the method of any one of Aspects 1-37, wherein during step b) 10% to 30% of a total mass of liquid, excluding the dialkyl malonate ester, is removed per hour.

Aspect 39 provides the method of Aspect 38, wherein 1.5% to 20% of the total mass of liquid present (for example, 1.5% to 18.5% or 1.0% to 15.3%) is removed per hour during step b) as the esterified malonate composition is formed.

Aspect 40 provides the method of any one of Aspects 38 or 39, wherein the removed liquid comprises a solvent that forms an azeotrope with water, wherein the solvent is different from the organic alcohol of step a).

Aspect 41 provides the method of Aspect 40, wherein the solvent comprises, toluene, benzene, hexane, cyclohexane, heptane, xylene, ethylbenzene, or a mixture thereof.

Aspect 42 provides the method of Aspect 41, wherein the solvent comprises toluene or cyclohexane.

Aspect 43 provides the method of any one of Aspects 41 or 42, further comprising adding recycled ethanol during step b).

Aspect 44 provides the method of any one of Aspects 1-43, wherein the method further comprises d) neutralizing inorganic acid present in the esterified malonate composition prior to step c).

Aspect 45 provides the method of any one of Aspects 1-44, wherein the distilling of the esterified malonate composition of step b) during step c) comprises a fractional distillation.

Aspect 46 provides the method of any one of Aspects 1-45, wherein the distillate of step c) comprises greater than 95 wt % dialkyl malonate ester based on the total amount of liquid present.

Aspect 47 provides the method of any one of Aspects 1-46, wherein the distillate of step c) comprises 95 wt % to 99.9 wt % dialkyl malonate ester based on the total amount of liquid present, 95 wt % to 99.5 wt % dialkyl malonate ester based on the total amount of liquid present, for example, at least 96 wt %, 97 wt %, 98 wt %, 99 wt %, or 99.9 wt % based on the total amount of liquid present.

Aspect 48 provides the method of any one of Aspects 1-47, wherein the distillate of step c) comprises greater than 95 wt % diethyl malonate ester or dimethyl malonate ester, preferably diethyl malonate ester.

Aspect 49 provides the method of any one of Aspects 1-48, wherein the distillate of step c) comprises 95 wt % to 99.9 wt % diethyl malonate ester based on the total amount of liquid present, 95 wt % to 99.5 wt % diethyl malonate ester based on the total amount of liquid present, for example, at least 96 wt %, 97 wt %, 98 wt %, 99 wt %, or 99.9 wt % based on the total amount of liquid present.

Aspect 50 provides the method of any one of Aspects 1-49, wherein the esterified malonate composition of step b) comprises 10 wt % to 99.9 wt % of Formula V (for example, from 70% to 99.5% or from 90% to 99.5%).

Aspect 51 provides the method of any one of Aspects 1-50, wherein the malonate composition of step a) is recovered from a fermentation process.

Aspect 52 provides the method of any one of Aspects 1-51, further comprising precipitating a portion of the malonate composition recovered from the fermentation process.

Aspect 53 provides a method for making a dialkyl malonate ester, the method comprising:
e) acidifying a malonate composition comprising:
   at least 30 wt % based on malonic acid equivalents of a compound of formula I:

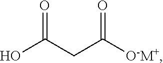

wherein M⁺ is a Group I alkali metal cation or ammonium cation, and wherein the malonate composition optionally further comprises at least one of:
a compound of formula II:

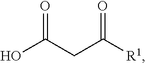

and malonic acid;
wherein the acidifying of the malonate composition comprises contacting an organic alcohol and an inorganic acid with the malonate composition to solubilize the malonate composition and to form a first acidified malonate composition comprising at least one of:
a compound of formula IV

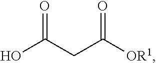

a compound of formula V

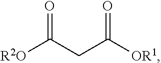

and
malonic acid,
wherein R¹ and R₂ are independently (C₁-C₈)alkyl;
f) heating the first acidified malonate composition to form an esterified malonate composition comprising the dialkyl malonate ester;
g) separating a precipitate comprising M⁺ from the esterified malonate composition;
h) distilling the esterified malonate composition of step g) to isolate a distillate comprising the dialkyl malonate ester, wherein the distillate comprises greater than or equal to 90 wt % dialkyl malonate ester (for example, 90 wt % to 100 wt % or 95 wt % to 99.9 wt % or 95 wt % to 99.5 wt %).

Aspect 54 provides the method of any one of Aspects 51 or 52, wherein the fermentation process is carried out a pH in a range of 3 to 5.

Aspect 55 provides the method of Aspect 54, wherein the dialkyl malonate ester comprises a (C₁-C₈)dialkyl malonate ester.

Aspect 56 provides the method of Aspect 55, wherein (C₁-C₈)dialkyl malonate ester comprises a (C₁-C₆)dialkyl malonate ester.

Aspect 57 provides the method of Aspect 56, wherein (C₁-C₆)dialkyl malonate ester comprises a (C₁-C₄)dialkyl malonate ester.

Aspect 58 provides the method of any one of Aspects 53-57 wherein the organic alcohol of step e) is methanol, ethanol, propanol(branched or linear), butanol(branched or linear), pentanol(branched, linear, or cyclic), hexanol(branched, linear, or cyclic), heptanol(branched or linear), or octanol(branched or linear).

Aspect 59 provides a method for making a dialkyl malonate ester, the method comprising:
i) acidifying a malonate composition comprising:
   at least 30 wt % based on malonic acid equivalents of a compound of formula I:

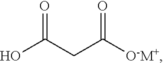

wherein M⁺ is a Group I alkali metal cation or ammonium cation, and wherein the malonate composition optionally further comprises at least one of:
a compound of formula II:

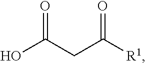

and malonic acid;
wherein the acidifying of the malonate composition comprises contacting an organic alcohol and an inorganic acid with the malonate composition to solubilize the malonate composition to form a first acidified malonate composition comprising at least one of:
a compound of formula IV

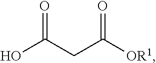

a compound of formula V

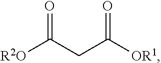

and
malonic acid,
wherein R¹ and R₂ are independently (C₁-C₈)alkyl; and
j) separating a precipitate comprising M⁺ from the first acidified malonate composition of step a) to form a second acidified malonate composition comprising formula IV, formula V, malonic acid, or a mixture thereof;
k) heating the second acidified malonate composition of j) to form an esterified malonate composition comprising the dialkyl malonate ester; and
l) distilling the esterified malonate composition of step k) to isolate a distillate comprising the dialkyl malonate ester, wherein the distillate comprises greater than or equal to 90 wt % dialkyl malonate ester (for example, 90 wt % to 100 wt % or 95 wt % to 99.9 wt % or 95 wt % to 99.5 wt %).

Aspect 60 provides the method of Aspect 59, wherein the dialkyl malonate ester comprises a (C₁-C₈)dialkyl malonate ester.

Aspect 61 provides the method of Aspect 60, wherein (C₁-C₈)dialkyl malonate ester comprises a C₁-C₆)dialkyl malonate ester.

Aspect 62 provides the method of Aspect 61, wherein (C₁-C₆)dialkyl malonate ester comprises a C₁-C₄)dialkyl malonate ester.

Aspect 63 provides the method of any one of Aspects 59-62 wherein the organic alcohol of step i) is methanol, ethanol, propanol(branched or linear), butanol(branched or linear), pentanol(branched, linear, or cyclic), hexanol(branched, linear, or cyclic), heptanol(branched or linear), or octanol(branched or linear).

Aspect 64 provides the method of any one of Aspects 1-63, wherein the organic alcohol is selected from the group consisting of methanol, ethanol, and propanol.

1.-53. (canceled)

54. A method for making a dialkyl malonate ester, the method comprising:
e) acidifying a malonate composition comprising:
at least 30 wt % based on malonic acid equivalents of a compound of formula I: 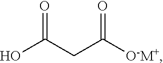
wherein M⁺ is a Group I alkali metal cation or ammonium cation, and wherein the malonate composition optionally further comprises at least one of:
a compound of formula II: 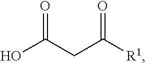
and malonic acid;
wherein the acidifying of the malonate composition comprises contacting an organic alcohol and an inorganic acid with the malonate composition to solubilize the malonate composition and to form a first acidified malonate composition comprising at least one of:
a compound of formula IV 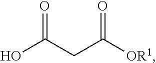
a compound of formula V 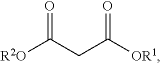
and
malonic acid,
wherein R¹ and R² are independently (C₁-C₈)alkyl;
f) heating the first acidified malonate composition to form an esterified malonate composition comprising the dialkyl malonate ester;
g) separating a precipitate comprising M⁺ from the esterified malonate composition;
h) distilling the esterified malonate composition of step g) to isolate a distillate comprising the dialkyl malonate ester, wherein the distillate comprises greater than or equal to 90 wt % dialkyl malonate ester (for example, 90 wt % to 100 wt % or 95 wt % to 99.9 wt % or 95 wt % to 99.5 wt %).

55. The method of claim 54, wherein the dialkyl malonate ester comprises a (C₁-C₈)dialkyl malonate ester.

56. The method of claim 55, wherein (C₁-C₈)dialkyl malonate ester comprises a (C₁-C₆)dialkyl malonate ester.

57. The method of claim 56, wherein (C₁-C₆)dialkyl malonate ester comprises a (C₁-C₄)dialkyl malonate ester.

58. The method claim 54 wherein the organic alcohol of step e) is methanol, ethanol, propanol(branched or linear), butanol(branched or linear), pentanol(branched, linear, or cyclic), hexanol(branched, linear, or cyclic), heptanol(branched or linear), or octanol(branched or linear).

59. A method for making a dialkyl malonate ester, the method comprising:
i) acidifying a malonate composition comprising:
at least 30 wt % based on malonic acid equivalents of a compound of formula I: 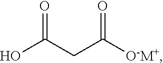
wherein M⁺ is a Group I alkali metal cation or ammonium cation, and wherein the malonate composition optionally further comprises at least one of:
a compound of formula II: 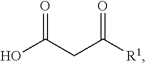
and malonic acid;
wherein the acidifying of the malonate composition comprises contacting an organic alcohol and an inorganic acid with the malonate composition to solubilize the malonate composition to form a first acidified malonate composition comprising at least one of:
a compound of formula IV 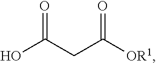
a compound of formula V 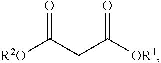
and
malonic acid,
wherein R¹ and R² are independently (C₁-C₈)alkyl; and
j) separating a precipitate comprising M⁺ from the first acidified malonate composition of step a) to form a second acidified malonate composition comprising formula IV, formula V, malonic acid, or a mixture thereof,
k) heating the second acidified malonate composition of j) to form an esterified malonate composition comprising the dialkyl malonate ester; and
l) distilling the esterified malonate composition of step k) to isolate a distillate comprising the dialkyl malonate ester, wherein the distillate comprises greater than or equal to 90 wt % dialkyl malonate ester (for example, 90 wt % to 100 wt % or 95 wt % to 99.9 wt % or 95 wt % to 99.5 wt %).

60. The method of claim 59, wherein the dialkyl malonate ester comprises a (C₁-C₈)dialkyl malonate ester.

61. The method of claim 60, wherein (C₁-C₈)dialkyl malonate ester comprises a (C₁-C₆)dialkyl malonate ester.

62. The method of claim 61, wherein (C₁-C₆)dialkyl malonate ester comprises a (C₁-C₄)dialkyl malonate ester.

63. The method of claim 59 wherein the organic alcohol of step i) is methanol, ethanol, propanol(branched or linear), butanol(branched or linear), pentanol(branched, linear, or cyclic), hexanol(branched, linear, or cyclic), heptanol(branched or linear), or octanol(branched or linear).

64. The method of claim 63, wherein the organic alcohol is selected from the group consisting of methanol, ethanol, and propanol.
